(19) United States
(12) Patent Application Publication
(10) Pub. No.: US 2009/0005787 A1
(43) Pub. Date: Jan. 1, 2009

(54) DEVICE AND SYSTEM FOR IMPLANTING POLYAXIAL BONE FASTENERS

(76) Inventors: ANGELA CRALL, Austin, TX (US); Larry O. Fisher, Smithville, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/770,560

(22) Filed: Jun. 28, 2007

(51) Int. Cl. A61B 17/04; A61B 17/56; A61B 17/58

(52) U.S. Cl. 606/104; 606/99; 606/305; 606/302

(57) ABSTRACT

A device, is provided for implanting in bony tissue a bone fastener rotatably connected to a collar. The sleeve is configured for threaded connection to the collar and has a central bore aligned with the longitudinal axis of the sleeve for receiving an inner shaft. The inner shaft inserts in the sleeve and has a distal end configured for connection to a portion of the bone fastener, such as an internal hex feature. In some embodiments a movable member may be inserted in an opening transverse the central bore and positionable to either rotatably or fixedly connect the inner shaft to the sleeve. In other embodiments, a collet nut threads onto a collet thread to radially compress sleeve tangs to frictionally engage inner shaft to sleeve.

### TECHNICAL FIELD OF THE DISCLOSURE

This disclosure relates generally to tools for implanting and removing screws in bony tissues within a body, and in particular to a device and system for retaining a driver in contact with a bone fastener head during surgical procedures.

### BACKGROUND

Bone may be subject to degeneration caused by trauma, disease, and/or aging. Degeneration may destabilize bone and affect surrounding structures. For example, destabilization of a spine may result in alteration of a natural spacing between adjacent vertebrae. Alteration of a natural spacing between adjacent vertebrae may subject nerves that pass between vertebral bodies to pressure. Pressure applied to the nerves may cause pain and/or nerve damage. Maintaining the natural spacing between vertebrae may reduce pressure applied to nerves that pass between vertebral bodies. A spinal stabilization procedure may be used to maintain the natural spacing between vertebrae and promote spinal stability.

Spinal stabilization may involve accessing a portion of the spine through soft tissue. Conventional stabilization systems may require a large incision and/or multiple incisions in the soft tissue to provide access to a portion of the spine to be stabilized. Conventional procedures may result in trauma to the soft tissue, for example, due to muscle stripping.

Spinal stabilization systems for a lumbar region of the spine may be inserted during a spinal stabilization procedure using a posterior spinal approach. Conventional systems and methods for posterolateral spinal fusion may involve dissecting and retracting soft tissue proximate the surgical site. Dissection and retraction of soft tissue may cause trauma to the soft tissue, and extend recovery time. Minimally invasive procedures and systems may reduce recovery time as well as trauma to the soft tissue surrounding a stabilization site.

Spinal stabilization and fixation devices such as rods may be coupled to vertebrae with pedicle screws. In an effort to reduce the number and size of incisions created during the implantation and removal processes, the drilling and implanting (e.g. threading) processes may be performed percutaneously. Thus, an incision may be made in the skin overlying the bone, and a trocar may be used to separate the soft tissue, creating a passage down to the implantation site. A drill may then be used to form a hole in the vertebra for the subsequent implantation of a bone fastener.

### SUMMARY OF THE DISCLOSURE

A spinal stabilization system may be installed in a patient to stabilize a portion of a spine. A spinal stabilization system may be installed using a minimally invasive procedure. An instrumentation kit may provide instruments and spinal stabilization system components necessary for forming a spinal stabilization system in a patient.

A spinal stabilization system may be used to achieve rigid pedicle fixation while minimizing the amount of damage to surrounding tissue. In some embodiments, a spinal stabilization system may be used to provide stability to two or more vertebrae. A spinal stabilization system may include an elongated member, two or more bone fastener assemblies, and/or a closure member. The bone fastener assembly may include, but is not limited to, a bone fastener and a collar. A first portion of the bone fastener may couple to a portion of the spine during use. A first portion of a collar may couple to a second portion of the bone fastener. A second portion of the collar may couple to an elongated member during use. In some embodiments, an orientation of the bone fastener may be independent of the orientation of the collar for a bone fastener assembly. After the bone fastener assembly is placed in a vertebral body, the collar coupled to the bone fastener may be further positioned so that the elongated member can be positioned in the collar and in at least one other collar that is coupled to another vertebral body by a bone fastener.

In an embodiment, a bone fastener assembly may include a bone fastener, a ring, and a collar. The ring may be positioned in the collar. Removal of the ring from the collar may be inhibited. A bone fastener may be positioned in the ring through a lower opening in the ring and in the collar. Splines of the bone fastener may be aligned with seats in the ring. The splines may be forced into the seats to couple the ring to the bone fastener. Separation of the ring from the bone fastener may be inhibited after the bone fastener is forced into the seats. The ring may angulate within the collar (i.e., the bone fastener may move relative to the collar within a defined range of motion) when the bone fastener assembly has been implanted in the vertebral body.

In an embodiment, a collar may include, but is not limited to, arms and a body. Arms and body of a collar may form a slot to receive an elongated member. When the elongated member is positioned in the collar, a portion of the elongated member may be coupled to a head of a bone fastener of the bone fastener assembly.

Inner surfaces of the arms of a bone fastener assembly collar may include a modified thread. The modified thread may engage a complementary modified thread of a closure member. A closure member may secure an elongated member to a bone fastener assembly. In some embodiments, a range of motion of a collar relative to a bone fastener may be skewed from a conical range of motion relative to a longitudinal center axis of the collar. The skew may be used to accommodate lordotic alignment and/or pedicle angle shift in adjacent vertebrae.

Different instruments may be used to form a spinal stabilization system in a patient using a minimally invasive procedure. The instruments may include, but are not limited to, positioning needles, guide wires, sleeves, bone fastener driver, mallets, tissue wedges, tissue retractors, tissue dilators, bone awls, taps, and an elongated member length estimator. An instrumentation kit may include, but is not limited to, two or more sleeves, a tissue wedge, an elongated member positioner, a counter torque wrench, an estimating tool, a seater, closure member driver, and/or combinations thereof.

Sleeves may be used during installation of one vertebral level stabilization systems at each of the two vertebrae to be stabilized. In an embodiment, a sleeve may be coupled to a collar of a bone fastener assembly, an inner shaft member may be coupled to a head portion of a bone fastener, and a movable member may be used to inhibit rotation of the inner shaft member relative to sleeve A shortcoming of prior art devices for implanting the bone fastener is that the device used to implant the bone fastener loosens its hold on the bone fastener. In some cases, the bone fastener disconnects from the tip of the device and must be retrieved from the patient's body, resulting in longer operating room time and possible internal damage to the patient. In some cases the bone fastener does not disconnect, but loosens such that the surgeon has less control over the whereabouts and direction of the tip of the bone fastener, resulting in missed attempts at threading the hole, or (if already partially threaded in the hole) the likelihood that the bone fastener will rotate off-axis and damage the hole.

An embodiment may have a sleeve that attaches to the collar and an internal shaft that attaches to the bone fastener, and which is lockable in the sleeve so the entire assembly rotates as one rigid unit.

Embodiments of the present disclosure enable surgeons to selectively configure an inner shaft and sleeve to either rotate independently or rotate as a single unit. Allowing the inner shaft and sleeve to rotate independently allows the surgeon to connect to, manipulate, and disconnect from either the bone fastener or the collar. Advantageously, embodiments of the present disclosure may be usefully applied to threading bone fasteners with attached collars in minimally invasive surgeries (MIS) due to the ease of manipulation of the inner shaft, sleeve, and movable member components. The inner shaft may be inserted and locked to the outer sleeve, and unlocked and removed from the outer sleeve using hand operations only (i.e., no other tools.) A further benefit to having an inner shaft that may be rigidly connected is that once the inner shaft has been locked to the outer shaft, the bone fastener and collar are retained in a rigid configuration that prevents partial disconnection that could lead to off-axis rotations (i.e. precession) by the bone fastener resulting in bony tissue damage and injury to the patient, or damage to the collar which could prevent proper attachment to the bone fastener or proper connection to a rod. Furthermore, partial disconnection may lead to complete disconnection from the collar or bone fastener, which could be harmful to the patient and extends the time in surgery to locate and retrieve a lost screw, bone fastener, or collar. In minimally invasive spine surgeries, the device must not accidentally disconnect from the screw, and equally important, the bone fastener must not partially disconnect or loosen from the device. A bone fastener that has loosened from the device could miss the target and hit the spinal cord, a disc, nerve, or artery, or damage the hole from the effects of precession such that implantation is not possible.

Advantageously, embodiments of the present disclosure provide methods in which an inner shaft and a sleeve rotate as a single assembled unit enable surgeons to surgically penetrate the patient at a selected site and percutaneously implant a collar and bone fastener an at the same time without fear of disconnection or loosening of either the bone fastener or the collar.

One embodiment of the present disclosure is generally directed to a device for implanting in bony tissue a bone fastener that is already connected to a collar. The device comprises a sleeve having a distal end configured for threaded connection to the collar, a bore aligned with the longitudinal axis of the outer sleeve, and a proximal end having at least one opening normal to the central bore. The device further comprises a movable member configured for slidable positioning in two positions in the at least one opening normal to the central bore, having an elongated pivot hole with a first section configured for rotatable connection to an inner shaft, and a second section configured for fixed connection to an inner shaft and communicably coupled to the first section. The device further comprises an inner shaft configured for insertion into the central bore of the sleeve such that the distal end, which is adapted with an attachment feature such as an internal hex or key-style feature for connection to a feature of the bone fastener may pass through the central bore of the outer sleeve, and a proximal end configured for selective engagement with a tool. The tool may be a handle or other tool for manual use, or may be a drill or other powered tool. Slidably positioning the movable member in the sleeve such that the inner shaft is in a first position axially aligned with the sleeve and the first section of the movable member maintains the inner shaft in rotatable connection with the outer sleeve, and slidably positioning the movable member in the sleeve such that the inner shaft is in a second position axially aligned with the sleeve and the second section of the movable member engages the inner shaft in fixed connection with the sleeve. When the device is attached to a bone fastener and collar and the movable member is in the second position such that the inner shaft is fixedly connected to the sleeve, the entire construct rotates as a single rigid unit to enable a surgeon to implant a bone fastener into bony tissue while the collar is already attached to the bone fastener.

Another embodiment is generally directed to a method comprising rotating a sleeve about its longitudinal axis such that a threaded portion of the distal end engages a threaded portion of the collar, slidably positioning a movable member in a first section of two positions in the sleeve opening normal to a bore, inserting an inner shaft at least partially into the bore of the sleeve such that the inner shaft is axially aligned axial to the sleeve, engaging the distal end of the inner shaft to a portion of the bone fastener, and moving the movable member from the first position to the second position such that the second section is fixed to the longitudinal axis of the sleeve.

Another embodiment is generally directed to a method comprising inserting a portion of the bone fastener in the collar and rotating a sleeve about its longitudinal axis such that a threaded portion of the distal end engages a threaded portion of the collar, slidably positioning a movable member in a first of two positions in the central bore, passing an inner shaft through the bore of the sleeve such that the inner shaft is axially aligned axial to the sleeve, engaging the distal end of the inner shaft to a portion of the bone fastener, then moving the movable member from the first position to the second position such that the inner shaft is fixed to the longitudinal axis of the outer sleeve. An incision in the patient may be entered with the bone fastener, collar, and a portion of the inner shaft and portion of the sleeve as a single unit and rotated as a rigid construct such that the bone fastener threads into bony tissue at a selected site. The method may further include slidably positioning the movable member from the second position to the first position such that the inner shaft is rotatably connected to the sleeve, rotating the sleeve in a reverse direction to disengage the distal end threads from the collar threads, and extracting the sleeve and inner shaft from the body, leaving the bone fastener and collar implanted in the bony tissue.

Another embodiment is directed to a device for implanting in bony tissue a bone fastener connected to a collar, having an inner shaft with a distal end configured for connection to a portion of the bone fastener and a proximal end configured for selective engagement with a tool. The inner shaft may be at least partially inserted into the central bore of a sleeve having a distal end configured for connection to a collar and a proximal end configured with a plurality of slots radially disposed about the longitudinal axis to form a plurality of proximally extending tangs, and a collet thread. A collet nut has a tapered inner surface and a thread for engagement with the collet thread, so that the action of threading the collet nut onto the collet thread radially compresses the plurality of tangs inward to frictionally engage the inner shaft, and the outer sleeve, inner shaft, bone fastener and collar rotate as a single rigid construct to implant the bone fastener in bony tissue with the collar attached.

Yet another embodiment is generally directed to a method comprising rotating a sleeve about its longitudinal axis such that a threaded portion of the distal end engages a threaded portion of the collar, inserting an inner shaft through the central bore of the sleeve aligned with the longitudinal axis of the outer sleeve, engaging the distal tip of the inner shaft with an attachment profile on the bone fastener, and threading a collet nut to engage the collet thread, such that the collet nut tapered inner surface contacts a plurality of tangs to inwardly flex the plurality of tangs such that the plurality of tangs frictionally engages the inner shaft.

Yet another embodiment is generally directed to a method comprising inserting a portion of a bone fastener having selected profile into a collar, threading the collar onto the distal end of a sleeve configured for attachment to a collar, such that the collar seats on the outer sleeve, inserting an inner shaft through the central bore of the sleeve aligned with the longitudinal axis of the outer sleeve, engaging the distal tip of the inner shaft with an attachment profile on the bone fastener, rotating a collet nut to engage a collet nut thread with the collet thread, and continued threading of the collet nut on the collet thread such that a collet nut tapered inner surface contacts a plurality of tangs to inwardly flex the plurality of tangs such that the inner shaft is frictionally connected to the sleeve. The method may further include surgically penetrating a body with the bone fastener, collar, and a portion of the inner shaft and portion of the sleeve, and rotating the inner shaft, and also the sleeve, collar, and bone fastener such that the bone fastener threads into bony tissue at a selected site. The method may further include rotating the collet nut in a reverse direction such that at least part of the collet nut thread disengages at least part of the collet thread to release the forces exerted by the collet nut tapered inner surface on the plurality of tangs, rotating the sleeve in the reverse direction such that the thread on the distal end of the sleeve disengages the threads on the collar, and withdrawing the portion of inner shaft and portion of the sleeve from the body.

The present disclosure overcomes prior art methods and systems for implanting threaded members in bony tissues with a design that allows one-handed operation of the locking mechanism. Embodiments of the present disclosure allow for ratcheting to circumvent the need for the surgeon to loosen, shift or remove a hand from the device during the implantation or removal processes.

These, and other, aspects of the disclosure will be better appreciated and understood when considered in conjunction with the following description and the accompanying drawings. The following description, while indicating various embodiments of the disclosure and numerous specific details thereof, is given by way of illustration and not of limitation. Many substitutions, modifications, additions or rearrangements may be made within the scope of the disclosure, and the disclosure includes all such substitutions, modifications, additions or rearrangements.

### BRIEF DESCRIPTION OF THE FIGURES

FIG. 1 depicts a perspective view of an embodiment of a spinal stabilization system.

FIG. 2 depicts a perspective view of an embodiment of a bone fastener assembly.

FIG. 3 depicts a perspective view of an embodiment of a bone fastener.

FIGS. 4A and 4B depict perspective views of embodiments of bone fastener assembly rings.

FIG. 5 depicts a perspective view of an embodiment of a bone fastener assembly collar.

FIG. 6 depicts a cross-sectional view of an embodiment of a bone fastener assembly.

FIGS. 7A-7C depict schematic views of a method of positioning a ring in a collar of a bone fastener assembly.

FIGS. 8A-8C show views of collar 112 and ring 110 during bottom loading insertion of the ring into the collar.

FIGS. 9A and 9B depict schematic views of positioning a bone fastener in a ring and collar to form a bone fastener assembly.

FIG. 10 depicts bone fastener assembly 102 with central axis 158 of collar 112 aligned with central axis 160 of bone fastener 108.

FIG. 11 depicts a perspective view of an embodiment of a closure member.

FIG. 12 depicts a cross-sectional representation of the closure member taken substantially along plane 15-15 indicated in FIG. 11.

FIG. 13 depicts a portion of a spinal stabilization system with closure member 106 coupled to collar 112 before tool portion 170 is sheared off.

FIG. 14A depicts a side view representation of an embodiment of a spinal stabilization system that utilizes a movable member to couple a sleeve and an inner shaft.

FIG. 14B depicts a cross-sectional view of a distal end of a sleeve and an inner shaft coupled to a bone fastener assembly in an embodiment of the present disclosure.

FIG. 14C depicts an enlarged view of the embodiment depicted in FIG. 14B.

FIG. 15A depicts a cross-sectional top view of an embodiment of a spinal stabilization system.

FIG. 15B depicts a cross-sectional top view of an embodiment of a spinal stabilization system in an alternate position.

FIG. 16 depicts an exploded view of a bone fastener assembly driver useful for implanting bone fastener assemblies.

FIG. 17 depicts a side view of an embodiment of a bone fastener assembly using a collet-style mechanism.

FIG. 18 depicts an exploded view of an embodiment of a bone fastener assembly using a collet-style mechanism.

FIG. 19 depicts a cross section view of a collet nut useful in a collet-style locking mechanism in an embodiment of a bone fastener assembly driver.

FIG. 20 depicts an end view of a collet nut useful in a collet-style locking mechanism in an embodiment of a bone fastener assembly driver.

FIG. 21 depicts a side view of a collet nut useful in a collet-style locking mechanism in an embodiment of a bone fastener assembly driver.

### DETAILED DESCRIPTION OF THE DISCLOSURE

The disclosure and the various features and advantageous details thereof are explained more fully with reference to the non-limiting embodiments that are illustrated in the accompanying drawings and detailed in the following description. Descriptions of well known starting materials, processing techniques, components and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, additions or rearrangements within the scope of the underlying inventive concept(s) will become apparent to those skilled in the art after reading this disclosure.

Reference is now made in detail to the exemplary embodiments of the disclosure, examples of which are illustrated in the accompanying drawings. Wherever possible, the same reference numbers will be used throughout the drawings to refer to the same or like parts (elements).

A spinal stabilization system may be installed in a patient to stabilize a portion of a spine. Spinal stabilization may be used, but is not limited to use, in patients having degenerative disc disease, spinal stenosis, spondylolisthesis, pseudoarthrosis, and/or spinal deformities; in patients having fracture or other vertebral trauma; and in patients after tumor resection. A spinal stabilization system may be installed using a minimally invasive procedure. An instrumentation set may include instruments and spinal stabilization system components for forming a spinal stabilization system in a patient.

A minimally invasive procedure may be used to limit an amount of trauma to soft tissue surrounding vertebrae that are to be stabilized. In some embodiments, the natural flexibility of skin and soft tissue may be used to limit the length and/or depth of an incision or incisions needed during the stabilization procedure. Minimally invasive procedures may provide limited direct visibility in vivo. Forming a spinal stabilization system using a minimally invasive procedure may include using tools to position system components in the body.

A minimally invasive procedure may be performed after installation of one or more spinal implants in a patient. The spinal implant or spinal implants may be inserted using an anterior procedure and/or a lateral procedure. The patient may be turned and a minimally invasive procedure may be used to install a posterior spinal stabilization system. A minimally invasive procedure for stabilizing the spine may be performed without prior insertion of one or more spinal implants in some patients. In some patients, a minimally invasive procedure may be used to install a spinal stabilization system after one or more spinal implants are inserted using a posterior spinal approach.

A spinal stabilization system may be used to achieve rigid pedicle fixation while minimizing the amount of damage to surrounding tissue. In some embodiments, a spinal stabilization system may be used to provide stability to two adjacent vertebrae (i.e., one vertebral level). A spinal stabilization system may include two bone fastener assemblies. One bone fastener assembly may be positioned in each of the vertebrae to be stabilized. An elongated member may be coupled and secured to the bone fastener assemblies. As used herein, “coupled” components may directly contact each other or may be separated by one or more intervening members. In some embodiments, a single spinal stabilization system may be installed in a patient. Such a system may be referred to as a unilateral, single-level stabilization system or a single-level, two-point stabilization system. In some embodiments, two spinal stabilization systems may be installed in a patient on opposite sides of a spine. Such a system may be referred to as a bilateral, single-level stabilization system or a single-level, four-point stabilization system.

In some embodiments, a spinal stabilization system may provide stability to three or more vertebrae (i.e., two or more vertebral levels). In a two vertebral level spinal stabilization system, the spinal stabilization system may include three bone fastener assemblies. One bone fastener assembly may be positioned in each of the vertebrae to be stabilized. An elongated member may be coupled and secured to the three bone fastener assemblies. In some embodiments, a single two-level spinal stabilization system may be installed in a patient. Such a system may be referred to as a unilateral, two-level stabilization system or a two-level, three-point stabilization system. In some embodiments, two three-point spinal stabilization systems may be installed in a patient on opposite sides of a spine. Such a system may be referred to as a bilateral, two-level stabilization system or a two-level, six-point stabilization system.

In some embodiments, combination systems may be installed. For example, a two-point stabilization system may be installed on one side of a spine, and a three-point stabilization system may be installed on the opposite side of the spine. The composite system may be referred to a five-point stabilization system.

Minimally invasive procedures may reduce trauma to soft tissue surrounding vertebrae that are to be stabilized. Only a small opening may need to be made in a patient. For example, for a single-level stabilization procedure on one side of the spine, the surgical procedure may be performed through a 2 cm to 4 cm incision formed in the skin of the patient. In some embodiments, the incision may be above and substantially between the vertebrae to be stabilized. In some embodiments, the incision may be above and between the vertebrae to be stabilized. In some embodiments, the incision may be above and substantially halfway between the vertebrae to be stabilized. Dilators, a targeting needle, and/or a tissue wedge may be used to provide access to the vertebrae to be stabilized without the need to form an incision with a scalpel through muscle and other tissue between the vertebrae to be stabilized. A minimally invasive procedure may reduce an amount of post-operative pain felt by a patient as compared to invasive spinal stabilization procedures. A minimally invasive procedure may reduce recovery time for the patient as compared to invasive spinal procedures.

Components of spinal stabilization systems may be made of materials including, but not limited to, titanium, titanium alloys, stainless steel, ceramics, and/or polymers. Some components of a spinal stabilization system may be autoclaved and/or chemically sterilized. Components that may not be autoclaved and/or chemically sterilized may be made of sterile materials. Components made of sterile materials may be placed in working relation to other sterile components during assembly of a spinal stabilization system.

Spinal stabilization systems may be used to correct problems in lumbar, thoracic, and/or cervical portions of a spine. Various embodiments of a spinal stabilization system may be used from the C1 vertebra to the sacrum. For example, a spinal stabilization system may be implanted posterior to the spine to maintain distraction between adjacent vertebral bodies in a lumbar portion of the spine.

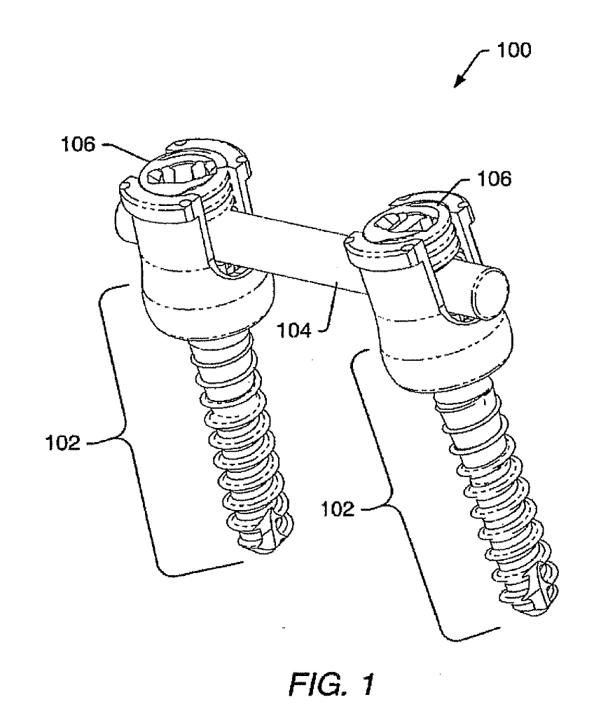

FIG. 1 depicts an embodiment of spinal stabilization system 100 that may be implanted using a minimally invasive surgical procedure. Spinal stabilization system 100 may include bone fastener assemblies 102, elongated member 104, and/or closure members 106. Other spinal stabilization system embodiments may include, but are not limited to, plates, dumbbell-shaped members, and/or transverse connectors. FIG. 1 depicts a spinal stabilization system for one vertebral level. In some embodiments, the spinal stabilization system of FIG. 1 may be used as a multi-level spinal stabilization system if one or more vertebrae are located between the vertebrae in which bone fastener assemblies 102 are placed. In other embodiments, multi-level spinal stabilization systems may include additional bone fastener assemblies to couple to one or more other vertebrae.

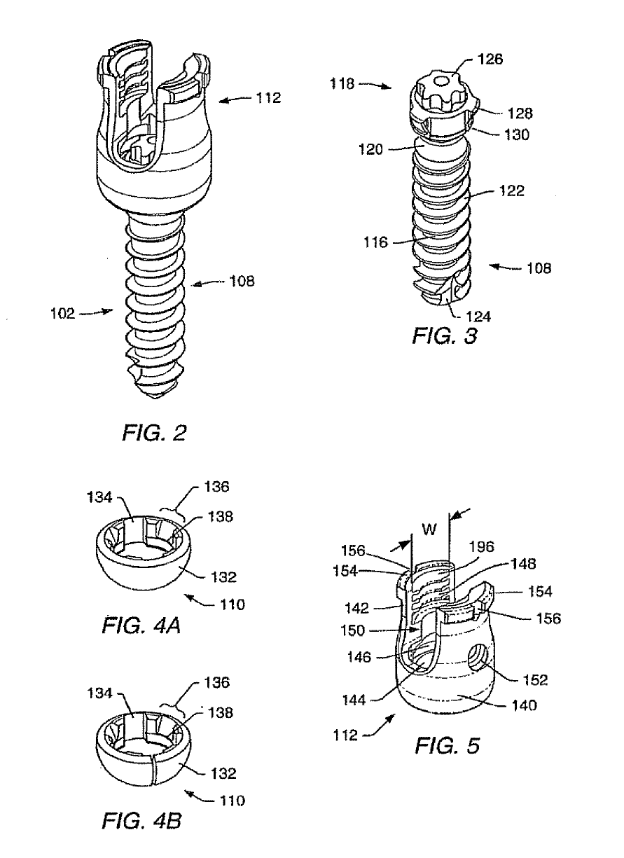

FIG. 2 depicts a perspective view of bone fastener assembly 102. FIG. 3, FIGS. 4A and 4B, and FIG. 5 depict embodiments of bone fastener assembly components. Components of bone fastener assembly 102 may include, but are not limited to, bone fastener 108 (shown in FIG. 3), ring 110 (shown in FIGS. 4A and 4B), and collar 112 (shown in FIG. 5). Bone fastener 108 may couple bone fastener assembly 102 to a vertebra. Ring 110 may be positioned between a head of bone fastener 108 and collar 112.

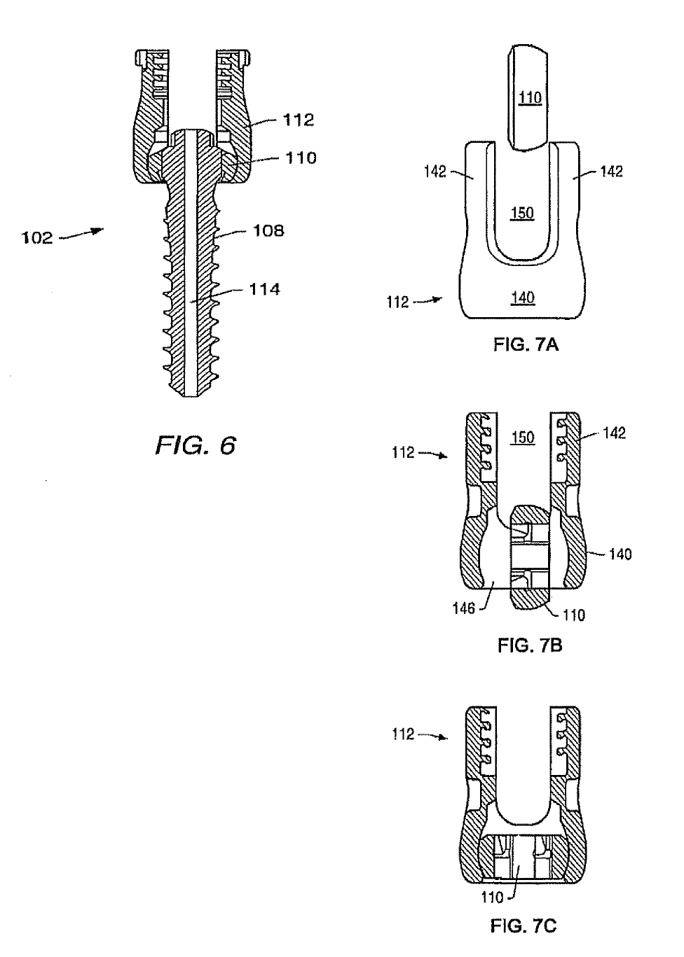

FIG. 6 depicts a cross-sectional representation of bone fastener 108, ring 110, and collar 112 of bone fastener assembly 102. Bone fastener 108 of bone fastener assembly 102 may include passage 114. Bone fastener 108 may be cannulated (i.e., passage 114 may run through the full length of the bone fastener). A guide wire may be placed through passage 114 so that bone fastener 108 may be inserted into a vertebra at a desired location and in a desired angular orientation relative to the vertebra with limited or no visibility of the vertebra.

A bone fastener may be, but is not limited to, a bone screw, a ring shank fastener, a barb, a nail, a brad, or a trocar. Bone fasteners and/or bone fastener assemblies may be provided in various lengths in an instrumentation set to accommodate variability in vertebral bodies. For example, an instrumentation set for stabilizing vertebrae in a lumbar region of the spine may include bone fastener assemblies with lengths ranging from about 30 mm to about 75 mm in 5 mm increments. A bone fastener assembly may be stamped with indicia (i.e., printing on a side of the collar). In some embodiments, a bone fastener assembly or a bone fastener may be color-coded to indicate a length of the bone fastener. In certain embodiments, a bone fastener with a 30 mm thread length may have a magenta color, a bone fastener with a 35 mm thread length may have an orange color, and a bone fastener with a 55 mm thread length may have a blue color. Other colors may be used as desired.

Each bone fastener provided in an instrumentation set may have substantially the same thread profile and thread pitch. In an embodiment, the thread may have about a 4 mm major diameter and about a 2.5 mm minor diameter with a cancellous thread profile. In certain embodiments, the minor diameter of the thread may be in a range from about 1.5 mm to about 4 mm or larger. In certain embodiments, the major diameter of the thread may be in a range from about 3.5 mm to about 6.5 mm or larger. Bone fasteners with other thread dimensions and/or thread profiles may also be used. A thread profile of the bone fasteners may allow bone purchase to be maximized when the bone fastener is positioned in vertebral bone.

FIG. 3 depicts an embodiment of bone fastener 108. Bone fastener 108 may include shank 116, head 118, and neck 120. Shank 116 may include threading 122. In some embodiments, threading 122 may include self-tapping start 124. Self-tapping start 124 may facilitate insertion of bone fastener 108 into vertebral bone.

Head 118 of bone fastener 108 may include various configurations to engage a driver that inserts the bone fastener into a vertebra. In some embodiments, the driver may also be used to remove an installed bone fastener from a vertebra. In some embodiments, head 118 may include one or more tool portions 126. Tool portions 126 may be recesses and/or protrusions designed to engage a portion of the driver. In some embodiments, bone fastener 108 may be cannulated for use in a minimally invasive procedure.

In an embodiment, head 118 of bone fastener 108 may have a generally smooth, spherical shape. In another embodiment, head 118 of bone fastener 108 may include one or more splines 128, as depicted in FIG. 3. In some head embodiments, head 118 may include three splines. Splines 128 may be equally spaced circumferentially around head 118 of bone fastener 108. In some head embodiments, splines 128 may be spaced at unequal distances circumferentially around head 118. Splines 128 may include various surface configurations and/or texturing to enhance coupling of bone fastener 108 with a ring of a bone fastener assembly. In some embodiments, sides of the splines may be tapered so that the splines form a dovetail connection with a ring. In some embodiments, spline width may be tapered so that a good interference connection is established when the bone bone fastener is coupled to a ring. Splines 128 may include one or more projections 130 to facilitate coupling bone fastener 108 with an inner surface of a ring. In some embodiments, projections 130 may be positioned on a lower portion of splines 128. In some embodiments, the splines may include recessed surfaces that accept projections extending from surfaces of the ring.

Neck 120 of bone fastener 108 may have a smaller diameter than adjacent portions of head 118 and shank 116. The diameter of neck 120 may fix the maximum angle that the collar of the bone fastener assembly can be rotated relative to bone fastener 108. In some embodiments, neck 120 may be sized to allow up to about 40 degrees or more of angulation of the collar relative to the bone fastener. In some embodiments, the neck may be sized to allow up to about 30 degrees of angulation of the collar relative to the bone fastener. In some embodiments, the neck may be sized to allow up to about 20 degrees of angulation of the collar relative to the bone fastener.

FIGS. 4A and 4B depict perspective views of embodiments of ring 110. Outer surface 132 of ring 110 may have a contour that substantially complements a contour of an inner surface of a collar in which the ring resides. A contour of the outer surface of the ring may be a spherical portion. When the ring is positioned in the collar, the complementary shape of the ring outer surface and the inner surface of the collar that contacts the ring allows angulation of the collar relative to a bone fastener coupled to the ring. The contour of the outer surface of the ring and the inner surface of the collar may inhibit removal of the ring from the collar after insertion of the ring into the collar.

Outer surface 132 of ring 110 may have a smooth finish. In some embodiments, outer surface 132 may be surface treated or include coatings and/or coverings. Surface treatments, coatings, and/or coverings may be used to adjust frictional and/or wear properties of the outer surface of the ring. In some embodiments, a portion of the outer surface of the ring may be shaped and/or textured to limit a range of motion of the collar relative to a bone fastener of a bone fastener assembly.

An inner surface of ring 110 may include one or more grooves 134 and/or one or more seats 136. Seats 136 may be circumferentially offset from grooves 134. Grooves 134 may be sized to allow passage of splines of a bone fastener (e.g., splines 128 shown in FIG. 3) through the ring. When the splines are inserted through grooves 134, the bone fastener may be rotated until the splines align with seats 136. The bone fastener may be pulled or driven so that the splines are positioned in seats 136. In some embodiments, projections (e.g., projections 130 in FIG. 3) may pass over ridges 138 of ring 110. Passage of the projections over ridges 138 may securely couple the bone fastener to the ring and inhibit separation of the ring from the bone fastener.

In a ring embodiment, a number of grooves 134 and a number of seats 136 may equal a number of splines 128 on a head of a bone fastener. Seats 136 and grooves 134 may be equally spaced circumferentially around the inner surface of ring 110. In some embodiments, seats 136 may be circumferentially offset about 60 degrees from grooves 134.

In some embodiments, as shown in FIG. 4A, a ring may be a complete ring without a split or slots. In some embodiments, a ring may include a split or slots to facilitate insertion of the ring into a collar. FIG. 4B depicts a ring with a split. In some embodiments, a ring with a split and/or slots may be compressed to ease insertion into a collar. Once positioned in the collar, the ring may expand to its original uncompressed dimensions, thus inhibiting removal from the collar.

As used herein, the term “collar” includes any element that wholly or partially encloses or receives one or more other elements. A collar may enclose or receive elements including, but not limited to, a bone fastener, a closure member, a ring, and/or an elongated member. In some embodiments, a collar may couple two or more other elements together (e.g., an elongated member and a bone fastener). A collar may have any of various physical forms. In some embodiments, a collar may have a “U” shape, however it is to be understood that a collar may also have other shapes.

A collar having a slot and an open top, such as collar 112 shown in FIG. 2 and in FIG. 5, may be referred to as an “open collar.” A bone fastener assembly that includes an open collar may be referred to as an “open fastener.” In some embodiments, an elongated member may be top loaded into the open fastener. A closure member may be coupled to the collar to secure the elongated member to the open fastener.

Collar 112 may include body 140 and arms 142. Arms 142 may extend from body 140. Body 140 of collar 112 may be greater in width than a width across arms 142 of collar 112 (i.e., body 140 may have a maximum effective outer diameter greater than a maximum effective outer diameter of arms 142). A reduced width across arms 142 may allow a sleeve to be coupled to the arms without substantially increasing a maximum effective outer diameter along a length of collar 112. Thus, a reduced width across arms 142 may reduce bulk at a surgical site.

A height of body 140 may range from about 3 millimeters (mm) to about 7 mm. In an embodiment, a height of body 140 is about 5 mm. Body 140 may include opening 144 in a lower surface of the body. To inhibit passage of a ring from collar 112, opening 144 may be smaller than an outer diameter of the ring. Inner surface 146 may be machined to complement a portion of an outer surface of a ring that is to be positioned in collar 112. Machining of inner surface 146 may enhance retention of a ring in collar 112. As used herein, the term “machining” refers to any mechanical, chemical, or thermal process used to form, shape, or finish a material, component, or structure useful in a bone bone fastenering assembly. Machining includes, but is not limited to, knurling, polishing, etching, bead blasting, layering, boring, and channeling. Inner surface 146 of body 140 may be complementary in shape to a portion of outer surface 132 of ring 110 (see FIGS. 4A-B) so that the ring is able to swivel in the collar. Inner surfaces and/or outer surfaces of collar 112 may be surface treated or include coatings and/or coverings to modify frictional properties or other properties of the collar.

Inner surfaces of arms 142 may include modified thread 148. Modified threads 148 may engage complementary modified threads of a closure member to secure an elongated member to a bone fastener assembly. Modified threads 148 may have a constant pitch or a variable pitch.

A height and a width of arms 142 may vary. Arms 142 may range in height from about 8 mm to about 15 mm. In an embodiment, a height of arms 142 is about 11 mm. A width (i.e., effective diameter) of arms 142 may range from about 5 mm to 14 mm. Arms 142 and body 140 may form slot 150. Slot 150 may be sized to receive an elongated member. Slot 150 may include, but is not limited to, an elongated opening of constant width, an elongated opening of variable width, a rectangular opening, a trapezoidal opening, a circular opening, a square opening, an ovoid opening, an egg-shaped opening, a tapered opening, and combinations and/or portions thereof. In some embodiments, a first portion of slot 150 may have different dimensions than a second portion of slot 150. In certain embodiments, a portion of slot 150 in first arm 142 may have different dimensions than a portion of slot 150 in second arm 142. When an elongated member is positioned in slot 150, a portion of the elongated member may contact a head of a bone fastener positioned in the collar.

In an embodiment of a collar, arms 142 of collar 112 may include one or more openings and/or indentions 152. Indentions 152 may vary in size and shape (e.g., circular, triangular, rectangular). Indentions 152 may be position markers and/or force application regions for instruments that perform reduction, compression, or distraction of adjacent vertebrae. In some embodiments, openings and/or indentions may be positioned in the body of the collar.

Arms 142 may include ridges or flanges 154. Flange 154 may allow collar 112 to be coupled to a sleeve so that translational motion of the collar relative to the sleeve is inhibited. Flanges 154 may also include notches 156. A movable member may pass into notch 156. When the movable member is positioned in notch 156, a channel in the sleeve may align with a slot in collar 112. With the movable member positioned in notch 156, rotational movement of collar 112 relative to the sleeve may be inhibited.

FIGS. 7A-7C show views of collar 112 and ring 110 during top loading insertion of the ring into the collar. Ring 110 may be positioned as shown in FIG. 7A and inserted past arms 142 into body 140. FIG. 7B depicts a cross-sectional view of ring 110 and collar 112 after insertion of the ring into the collar through slot 150. After insertion of ring 110 into collar 112, the ring may be rotated so that a bone fastener may be positioned through the ring. FIG. 7C depicts a cross-sectional view of ring 110 and collar 112 after rotation of the ring in the collar.

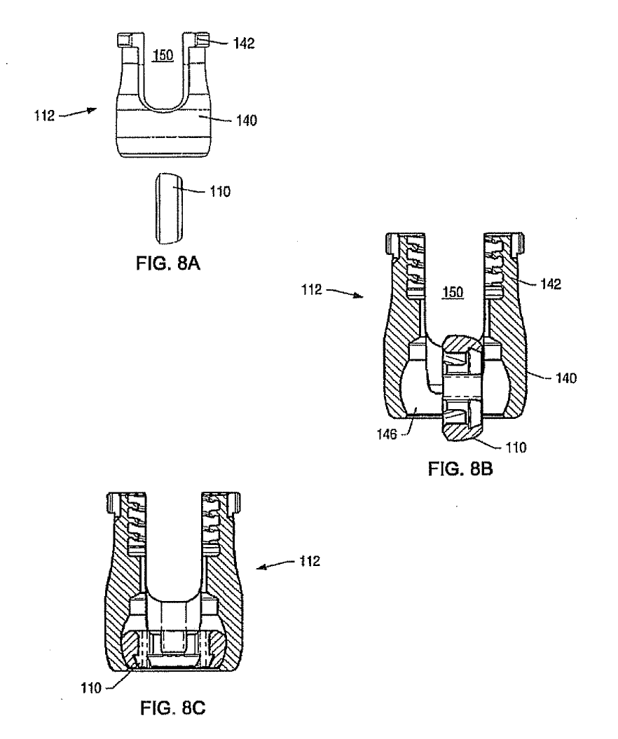

FIGS. 8A-8C show views of collar 112 and ring 110 during bottom loading insertion of the ring into the collar. Ring 110 may be positioned as shown in FIG. 8A and inserted into body 140 through an opening in the bottom of collar 112. In some embodiments, ring 110 may be inserted into body 140 through a groove or a slot in the bottom of collar 112. In certain embodiments, collar 112 designed for bottom insertion of ring 110 may have narrower slot 150 than a collar designed for top insertion of a ring. Collar 112 with narrower slot 150 may allow an elongated member with a reduced diameter to be used in a spinal stabilization system. Collar 112 with narrower slot 150 may be used to reduce bulk at a surgical site.

FIG. 8B depicts a cross-sectional view of ring 110 and collar 112 after insertion of the ring into the collar through the opening in the bottom of the collar. After insertion of ring 110 into collar 112, the ring may be rotated so that a bone fastener may be positioned through the ring. Tolerance between an outer surface of ring 110 and an inner surface of body 140 shown in FIGS. 7A-7C and 8A-8C may require force to be applied to the ring to drive the ring into the body. Once ring 110 is positioned in body 140, the ring may expand slightly. In certain embodiments, significant force may be required to remove ring 110 from body 140 (i.e., the ring may be substantially unreleasable from the body). The required force may inhibit unintentional removal of ring 110 from body 140. FIG. 8C depicts a cross-sectional view of ring 110 and collar 112 after rotation of the ring in the collar.

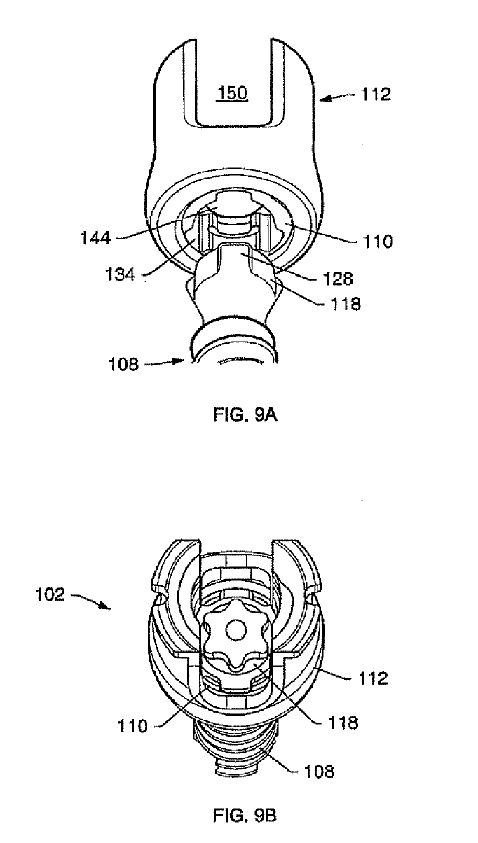

FIG. 9A depicts bone fastener 108 before insertion of the bone fastener into ring 110 positioned in collar 112. Splines 128 may be aligned with grooves 134 to allow passage of head 118 through ring 110 and into collar 112. FIG. 9B depicts bone fastener 108, ring 110, and collar 112 after the bone fastener has been rotated and head 118 has been coupled to seats in the ring to form bone fastener assembly 102. Inserting bone fastener 108 through opening 144 in collar 112 (depicted in FIG. 9A) may allow use of bone fasteners that have shanks and/or heads with larger diameters than can pass through slot 150. Bone fasteners with large diameter shanks may form a bone fastener assembly (threaded or otherwise) that securely fastens to vertebral bone during use.

A bone fastener may be rotatably positioned in a collar such that the bone fastener is able to move radially and/or rotationally relative to the collar (or the collar relative to the bone fastener) within a defined range of motion. The range of motion may be provided within a plane, such as by a hinged connection, or within a three-dimensional region, such as by a ball and socket connection. Motion of the bone fastener relative to the collar (or the collar relative to the bone fastener) may be referred to as “angulation” and/or “polyaxial movement”. The systems and methods of the present disclosure enable surgeons and other medical professionals to “lock” the bone fastener to the collar during implantation. When a bone fastener is loosely connected to the collar, the bone fastener is able to rotate off-axis. This motion, known as precession, is undesirable in surgery due to the undesirable possibility of damage to the tissue in which the bone fastener is intended such that implantation is not possible, or damage to surrounding tissue, which may include the spinal cord, arteries, or other organs and tissue.

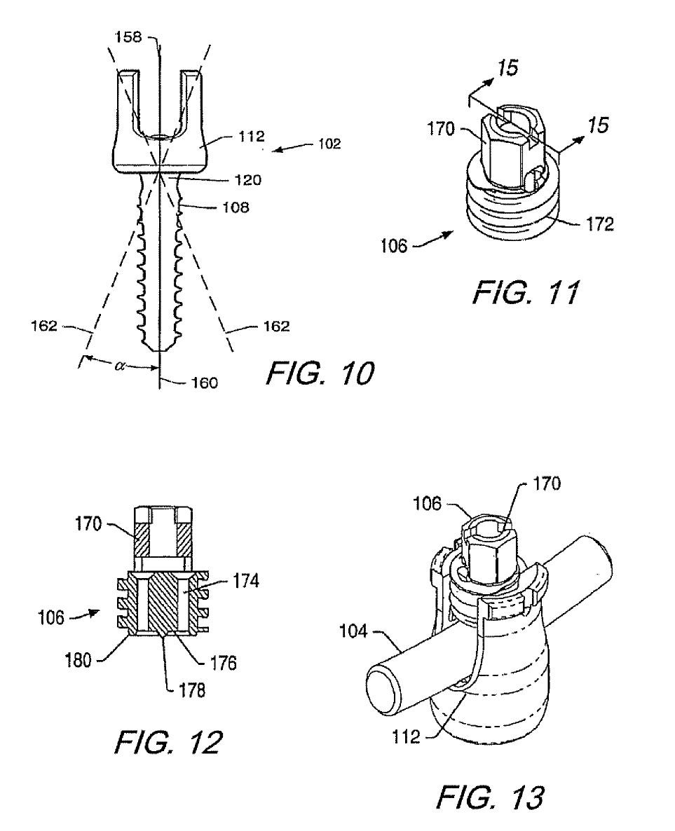

FIG. 10 depicts bone fastener assembly 102 with central axis 158 of collar 112 aligned with central axis 160 of bone fastener 108. Bone fastener 108 may be able to angulate in a symmetrical conical range of motion characterized by angle alpha about the aligned axes. Bone fastener 108 may be constrained from motion outside of limit axis 162 by contact between neck 120 of bone fastener 108 and collar 112. Alignment of axis 160 of bone fastener 108 with central axis 158 of collar 112 may be considered a neutral position relative to the range of motion. The alignment is a neutral position because bone fastener 108 may angulate an equal amount in any direction from central axis 158. When a driver is inserted into bone fastener 108, axis 160 of bone fastener 108 may be substantially aligned with axis 158 of collar 112 to facilitate insertion of the bone fastener into a vertebral body.

A closure member may be coupled to a collar of a bone fastener assembly to fix an elongated member positioned in the collar to the bone fastener assembly. In some embodiments, a closure member may be cannulated. In certain embodiments, a closure member may have a solid central core. A closure member with a solid central core may allow more contact area between the closure member and a driver used to couple the closure member to the collar. A closure member with a solid central core may provide a more secure connection to an elongated member than a cannulated closure member by providing contact against the elongated member at a central portion of the closure member as well as near an edge of the closure member.

FIG. 11 depicts closure member 106 prior to insertion of the closure member into a collar of a bone fastener assembly. Closure member 106 may include tool portion 170 and male modified thread 172. Tool portion 170 may couple to a tool that allows closure member 106 to be positioned in a collar. Tool portion 170 may include various configurations (e.g., threads, hexalobular connections, hexes) for engaging a tool (e.g., a driver). Male modified thread 172 may have a shape that complements the shape of a female modified thread in arms of a collar (e.g., modified thread 148 depicted in FIG. 5).

FIG. 12 depicts a cross-sectional representation of closure member 106 taken substantially along plane 15-15 of FIG. 11. Closure member 106 may include removal openings 174. A drive tool may be inserted into removal openings 174 to allow removal of closure member 106 after tool portion 170 has been sheared off. Removal openings 174 may include any of a variety of features including, but not limited to, sockets, holes, slots, and/or combinations thereof. In an embodiment, removal openings 174 are holes that pass through bottom surface 176 of closure member 106.

A bottom surface of a closure member may include structure and/or texturing that promotes contact between the closure member and an elongated member. A portion of the structure and/or texturing may enter and/or deform an elongated member when the closure member is coupled to the elongated member. Having a portion of the closure member enter and/or deform the elongated member may couple the elongated member to the closure member and a bone fastener assembly so that movement of the elongated member relative to the bone fastener assembly is inhibited. In a closure member embodiment, such as the embodiment depicted in FIG. 12, bottom surface 176 of closure member 106 may include point 178 and rim 180. In some embodiments, rim 180 may come to a sharp point. In some embodiments, a height of rim 180 may be less than a height of point 178. In other embodiments, a height of rim 180 may be the same or larger than a height of point 178. In some embodiments, rim 180 may not extend completely around the closure member. For example, eight or more portions of rim 180 may be equally spaced circumferentially around closure member 106. In certain embodiments, a solid central core including point 178 and rim 180 may enhance the ability of closure member 106 to secure an elongated member in a collar.

FIG. 13 depicts a portion of a spinal stabilization system with closure member 106 coupled to collar 112 before tool portion 170 is sheared off. Closure member 106 may couple to collar 112 by a variety of systems including, but not limited to, standard threads, modified threads, reverse angle threads, buttress threads, or helical flanges. A buttress thread on a closure member may include a rearward-facing surface that is substantially perpendicular to the axis of the closure member. Closure member 106 may be advanced into an opening in a collar to engage a portion of elongated member 104. In some embodiments, closure member 106 may inhibit movement of elongated member 104 relative to collar 112.

Various instruments may be used in a minimally invasive procedure to form a spinal stabilization system in a patient. The instruments may include, but are not limited to, positioning needles, guide wires, dilators, bone awls, bone taps, sleeves, bone fastener drivers, bone fastener assembly drivers, tissue wedges, elongated member length estimating tools, mallets, tissue retractors, and tissue dilators. The instruments may be provided in an instrumentation set. The instrumentation set may also include components of the spinal stabilization system. The components of the spinal stabilization system may include, but are not limited to, bone fastener assemblies of various sizes and/or lengths, elongated members, and closure members.

Instruments used to install a spinal stabilization system may be made of materials including, but not limited to, stainless steel, titanium, titanium alloys, ceramics, and/or polymers. Some instruments may be autoclaved and/or chemically sterilized. Some instruments may include components that cannot be autoclaved or chemically sterilized. Components of instruments that cannot be autoclaved or chemically sterilized may be made of sterile materials. The sterile materials may be placed in working relation to other parts of the instrument that have been sterilized.

A bone fastener assembly driver may be used to install bone fastener assemblies in vertebral bone. A bone fastener assembly driver may include a sleeve to couple to a collar of a bone fastener assembly. A distal end of a sleeve may be tapered or angled to reduce bulk at a surgical site. Instruments may be inserted into the sleeve to manipulate the bone fastener assembly. Movement of the sleeve may alter an orientation of a collar relative to a bone fastener of the bone fastener assembly. In some embodiments, a sleeve may be used as a retractor during a spinal stabilization procedure. Instruments may access a bone fastener assembly through a passage in a sleeve, such as a central bore extending longitudinally through the sleeve. A sleeve may have one or more openings positioned transverse the central bore. In an embodiment, a movable member may be inserted through the transverse openings in the sleeve. An inner shaft may be inserted in the central bore of the sleeve and through a portion of the movable member. A movable member may be positioned to engage the inner shaft to inhibit rotation of the inner shaft relative to the sleeve or may be positioned to allow the inner shaft to rotate inside the sleeve. The outer surface of a movable member may be flat, curved, or angled. In some embodiments, the outer surface of a movable member may be oval. In some embodiments, the outer surface of an inner shaft and/or an inner surface of a movable member may be textured to inhibit rotation of the inner shaft relative to the sleeve. In certain embodiments, a proximal end of an inner shaft may include a tool engaging portion. A tool engaging portion may include, but is not limited to, a hex section, a hexalobular section, a tapered section, a bead, a knot, a keyed opening, a coating, a threading, and/or a roughened surface for engaging a drive that rotates or otherwise displaces the bone fastener.

A cross section relative to a longitudinal axis of a sleeve may have shapes including, but not limited to, circular, ovoid, square, pentagonal, hexagonal, and combinations thereof. In certain embodiments, a thickness or width of a sleeve may be uniform. In certain embodiments, a thickness or width of a sleeve may vary along the length of the sleeve.

Embodiments of sleeves may be coupled to bone fastener assemblies in various configurations. In some embodiments an elongated member seated in the collar of the bone fastener assembly would lie below a distal end of sleeve. Having the elongated member below the distal end of sleeve may reduce bulk at the surgical site. With the distal end of the sleeve positioned above the elongated member, interference of the secured elongated member with the sleeve may be avoided during removal of the sleeve.

In some embodiments, a sleeve flange may engage a flange on the collar to inhibit translation of the sleeve relative to the collar of a bone fastener assembly. In some sleeve and collar coupling embodiments, the sleeve and the collar may include members that work together to inhibit radial expansion of walls of the sleeve.

In some sleeve and collar coupling embodiments, a sleeve may include a protrusion that mates with a complementary groove in a collar. Alternatively, a sleeve may include a groove that mates with a complementary protrusion of a collar.

Sleeves may be of various lengths. Sleeves of different lengths may be used in the same surgical procedure. A sleeve length used in a spinal stabilization procedure may be determined by a patient's anatomy. Sleeves may be just short enough to allow manipulation by a medical practitioner above an incision in a patient. Sleeves that are too long may require a longer incision and/or a larger tissue plane for insertion of a spinal stabilization system. Sleeves with excess length may be bulky and hard to manipulate during a surgical procedure.

A sleeve may be flexible over its entire length or include a flexible portion near a proximal end of the sleeve. A flexible portion may allow positioning of a proximal portion of a sleeve in a desired location. A flexible portion may be produced from any of various materials including, but not limited to, a surgical grade plastic, rubber, or metal. A flexible portion may be formed of various elements, including, but not limited to, a tube, a channel, or a plurality of linked segments. A sleeve, such as sleeve 244, may be flexible when used alone, but may connect to one or more of collar, bone fastener, and inner shaft to result in a rigid unit for implanting bone fasteners.

After a bone fastener assembly is coupled to a sleeve, an inner shaft may be coupled to a bone fastener of the bone fastener assembly. The inner shaft, coupled to the sleeve and collar, may be used to insert the bone fastener assembly into vertebral bone. When polyaxial bone fastener assemblies are positioned in vertebral bone, sleeves coupled to collars of the bone fastener assemblies may be moved in desired positions. FIG. 22 depicts a cross-sectional view of a portion of a bone fastener assembly driver. In an embodiment such as depicted in FIG. 22, a bone fastener 108 may be seated inside a collar 112. A thread 254 of a sleeve 244 may engage threads 148 on collar 112. A driver on distal end 259 of inner shaft 231 may engage tool portions 126 on bone fastener 108. In this configuration, inner shaft 231 and bone fastener 108 may rotate independent of collar 112 and/or sleeve 244 unless sleeve 244 and inner shaft 231 are engaged at their respective proximal ends.

In some embodiments, clearance between the inner shaft and the sleeve may be relatively small. The small clearances may inhibit undesired movement of the instruments relative to each other and/or reduce bulkiness at the surgical site.

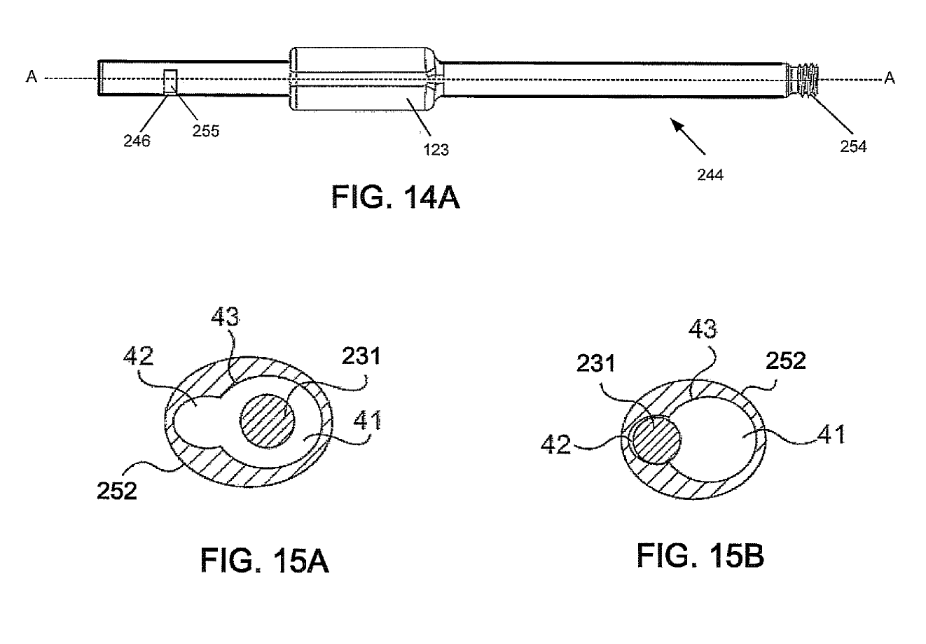

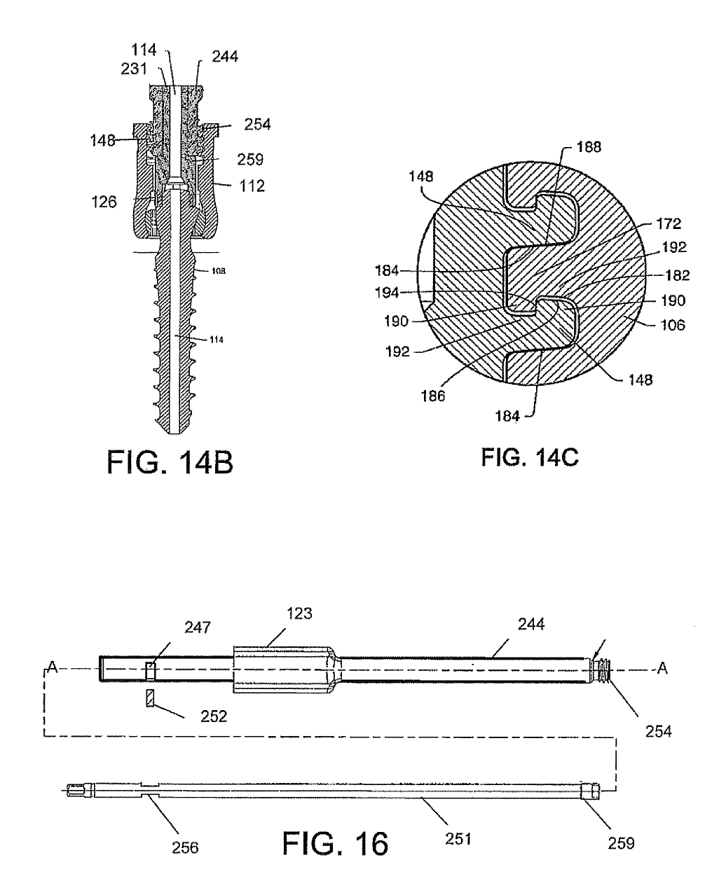

FIG. 14B depicts a cross-sectional view of a distal end of a sleeve 244 and an inner shaft 251 coupled to bone fastener assembly 102. Sleeve 244 may include male modified thread 172. Male modified thread 172 may include male distal surface 182 and male proximal surface 184, as shown in FIG. 14B. Collar 112 may include female modified thread 148 on an inside surface of arms 142. Female modified thread 148 may include female proximal surface 186 and female distal surface 188.

FIG. 14C depicts an enlarged view of a portion of the view in FIG. 14B. Raised portions 190 and recessed portions 192 may be included on male distal surface 182 and female proximal surface 186. Cooperating surfaces 194 of modified threads 172 and 148 may contact or be proximate to one another during use. As used herein, “proximate” means near to or closer to one portion of a component than another portion of a component. Engagement of cooperating surfaces 194 of modified threads 172 and 148 during use may inhibit radial expansion of collar 112. Engagement of cooperating surfaces 194 may inhibit spreading of arms 142 away from each other (i.e., inhibit separation of the arms). In some embodiments, cooperating surfaces 194 may be substantially parallel to a central axis of closure member 106. In other embodiments, cooperating surfaces 194 may be angled relative to a central axis of sleeve 244.

In an embodiment, a bone fastener assembly and a bone fastener assembly driver may be coupled with a running fit. A running fit (i.e., a fit in which parts are free to rotate) may result in predictable loading characteristics of a coupling of a bone fastener assembly and a closure member.

In an embodiment, a position (i.e., axial position and angular orientation) of a modified thread of a collar may be controlled, or “timed,” relative to selected surfaces of the collar. For example, a modified thread form may be controlled relative to a top surface of a collar and an angular orientation of the slots of the collar. In some embodiments, positions of engaging structural elements of other coupling systems (e.g., thread forms) may be controlled.

Controlling a position of a modified thread form may affect a thickness of a top modified thread portion of a collar. In FIG. 5, top modified thread portion 196 is the first modified thread portion to engage a bone fastener assembly driver. In an embodiment, a position of a modified thread form may be selected such that the thickness of the leading edge of a top modified thread portion is substantially equal to the full thickness of the rest of the modified thread.

Controlling a position of a modified thread form of a collar may increase a combined strength of engaged modified thread portions for a collar of a given size (e.g., wall height, modified thread dimensions, and thread pitch). Controlling a position of the modified thread form may reduce a probability of failure of modified thread portions, and thus reduce a probability of coupling failure between a collar and a bone fastener assembly driver.

If a thickness of a modified thread portion of a given size and profile is reduced below a minimum thickness, the modified thread portion may not significantly contribute to the holding strength of the modified thread of a collar. In an embodiment, a position of a modified thread form of a collar may be controlled such that a thickness of a top modified thread portion is sufficient for the portion to increase a holding strength of the collar. In one embodiment, a top modified thread portion may have a leading edge thickness of about 0.2 mm.

In an embodiment, a position of a modified thread form of a collar may be selected to ensure that a bone fastener assembly driver engages a selected minimum number of modified thread portions on each arm of the collar. In an embodiment, at least two modified thread portions having a full thickness over width w of a collar arm (shown in FIG. 5) may be engaged by a bone fastener assembly driver at each arm. Alternatively, a bone fastener assembly driver may engage parts of three or more modified thread portions on each arm, with the total width of the portions equal to at least two full-width portions. Allowances may be made for tolerances in the components (e.g., diameter of the elongated member) and/or anticipated misalignment between the components, such as misalignment between an elongated member and a slot. In an embodiment, a substantially equal number of modified thread portions in each arm may engage the bone fastener assembly driver when an elongated member is coupled to a bone fastener assembly.

In some embodiments, a movable member may be inserted in one or more openings 255 in wall 246 of sleeve 244. An inner shaft may be inserted through a first section or a second section of a movable member. The first section and second section may be positioned such that a central axis of the first section or the second section is axially aligned with a sleeve and the longitudinal axis of the inner shaft. In some configurations, the inner shaft and a sleeve may rotate independently, and in other configurations may rotate as a single unit. Allowing the inner shaft and sleeve to rotate independently allows the surgeon to connect to, manipulate, and disconnect from either the bone fastener or the collar. Advantageously, embodiments of the present disclosure may be usefully applied to threading bone screws with attached collars in minimally invasive surgeries (MIS) due to the ease of manipulation of the device attachment. For example, distal end of the sleeve may be tapered to reduce bulk (e.g., reduce spin diameter) at a surgical site. A distal end of sleeve may include a flange that mates with a complementary flange on a collar of a bone fastener assembly. The inner shaft may be inserted and locked to the sleeve, and unlocked and removed from the outer shaft using hand operations only (i.e., no other tools.)

FIGS. 15A-B depict top views of an embodiment of movable member 252 configured for insertion into to a sleeve of a bone fastener assembly driver. Movable member 252 has a first section 41 and a second section 42. First section 41 may be sized and shaped such that inner shaft 251 can rotate when passing through it, Second section 42 may be sized and shaped so that inner shaft 251 cannot rotate relative to movable member 252 when inner shaft 251 passes through second section 42. When movable member 252 is positioned in an outer sleeve, inner shaft 251 may be either free to rotate in first section 41 independent of the sleeve or fixed in second section 42 and therefore may only rotate with the outer sleeve. First section 41 and second section 42 may be communicably coupled to form elongated hole 43 such that movable member 252 may be positioned in one of two positions about inner shaft 251 such that inner shaft 251 passes through either first section 41 or second section 42.

Movable member 252 may be positionable in opening 255 in sleeve 244 such that a first section is axially aligned with bore 250 in sleeve 244 to enable inner shaft 251 to rotate inside sleeve 244. Movable member 252 may be positionable in opening 255 in sleeve 244 such that a second section is axially aligned with bore 250 in sleeve 244 to enable rotation of shaft 251 and sleeve 244 as a single unit. Movable member 252 may have a rectilinear or curvilinear exterior outer surface for selected contact with opening 255 and bore 250 of sleeve 244. In preferred embodiments, the outer surface of movable member 252 may be generally an oval, which advantageously enables movable member 252 to compress slightly for insertion into opening 255 and requires only one opening 247 normal to bore 250 because the curved surface allows movable member 252 to be easily aligned with the longitudinal axis of bore 250.

To enable both inner shaft 251 and sleeve 244 to rotate as a single unit, thread 254 of sleeve 244 may be threadably engaged to threads on collar 112 and the driver tip of shaft 251 may be engaged with the head of bone fastener 108. Movable member 252 may be moved to a second position, which aligns the second section 42 of movable member 252 with inner shaft 231 and the axis for inner shaft 231 with the longitudinal axis of sleeve 244, thereby inhibiting shaft 251 from rotating independently of sleeve 244 while aligning the axes. This configuration may enable the surgeon to implant bone fastener 108 in bony tissue while it is connected to collar 112.

Movable member 252 may be configured such that when second section 42 of movable member is axially aligned with bore 250 of sleeve 244, a portion of movable member 252 is flush with sleeve 244, and text or symbols may indicate the state of movable member 252. Advantageously, having movable member 252 flush with sleeve 244 and having text or other symbols to indicate the state of movable member 252 provide visual and tactile indication to the surgeon that the movable member 252 is positioned correctly and that shaft 251 is fixedly connected to sleeve 244. In other words, if no portion of movable member 252 is flush with sleeve 244, or no symbol is visible to indicate the state of movable member 252, the surgeon may know that movable member 252 is not properly in place and that the assembly may not be in the desired configuration.

FIG. 16 depicts an exploded view of a bone fastener assembly driver useful for implanting bone fastener assemblies. Sleeve 244 may be coupled to a collar of the bone fastener assembly and may further be inhibited from rotating relative to inner shaft 251 using movable member 252. Inner shaft 251 and other instruments may be inserted through bore 250 of sleeve 244 to access an anchored bone fastener assembly coupled to the sleeve 244.

Inner shaft 251 may have splines, flattened areas, or other features for coupling or engaging with movable member 252. In the embodiment depicted in FIG. 16, inner shaft 251 may have flattened areas 256 sized, shaped, or otherwise configured for engagement with movable member 252.

A sleeve and inner shaft may be coupled to a bone fastener assembly in various ways to inhibit movement of the sleeve relative to the inner shaft, and further to a collar of the bone fastener assembly. A system used to couple the sleeve to the bone fastener assembly may inhibit rotation and translation of the sleeve relative to the inner shaft and collar.

In an embodiment of a collet-style connection system, a sleeve may include a radial array of proximally extending deflectable arms. The deflectable arms may be forced inward during rotation of a sleeve about the longitudinal axis of the sleeve. When the collet mechanism is seated to the sleeve, the deflectable arms may be positioned against an inner shaft in the bore of the sleeve. The friction force of the deflectable arms against the inner shaft may inhibit rotation and translation of the sleeve relative to the sleeve. Separation of the sleeve from the collar may be achieved by counter-rotating the collet mechanism to allow the deflectable arms to return to an undeflected state.

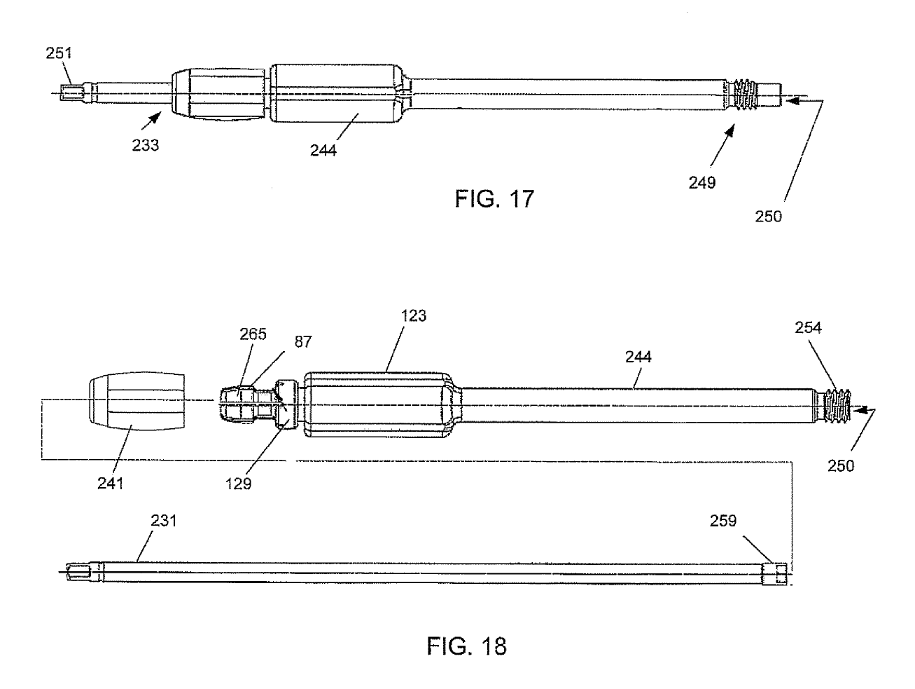

FIG. 17 depicts a side view of an embodiment of a bone fastener assembly using a collet-style mechanism. Inner shaft 251 may be inserted into central bore 250 of sleeve 244 through distal end 249 or proximal end 233.

FIG. 18 depicts an exploded side view of a bone fastener assembly 102 having an inner shaft 251 inside a sleeve 244. When inner shaft 251 is inside central bore 250 of sleeve 244, rotation of inner shaft 251 may be inhibited by collet-type locking mechanism 283. When collet-style locking mechanism 283 engages inner shaft 251 and proximal end 253 of inner shaft 251 is rotated, both inner shaft 251 and sleeve 244 (and therefore distal ends 257 and 249) rotate as a single unit to implant a bone fastener into bony tissue, with the bone fastener already attached to a collar.

Sleeve 244 may further include hand interfaces for tactile sensation, grip, or mechanical advantage for the surgeon, such as hand interface 123, and may further include writing 125 or other identifying marks or symbols. Hand interface 123 may be formed by adding or layering material onto sleeve 244, by boring, cutting, or otherwise removing material from sleeve 244, by machining (e.g. knurling, bead blasting, or etching) material on sleeve 244, or some combination. Hand interface 123 may be positioned anywhere along sleeve 244 to provide better grip, reduce hand strain, increase the rotational mechanical advantage, or otherwise benefit the surgeon. For example, hand interface 123 may be positioned closer to the proximal end of sleeve 244 than collet-style mechanism 140.

Sleeve 244 may have a distal end 249 having threads with a selected profile, for example, a 60 degree thread with a 0.066 pitch for rotatable engagement with threads on a collar, a proximal end having a plurality of proximally extending tangs 265 formed by cutting a plurality of slots 87 radially disposed about the longitudinal axis of sleeve 244 or otherwise formed, and a collet thread 129 having selected thread length and thread count, for example ½-20 UNF-2A.

Sleeve 244 may further include hand interface 123 for tactile sensation, grip, or mechanical advantage for the surgeon. Hand interface 123 may have selected geometry, which may provide better grip for the surgeon, less muscle strain, easy hand positioning, and more leverage for greater torque application by the surgeon. Hand interface 123 may include knurling, bead blasting, or other surface treatments, or adding ridges, convexities, projections, or other adaptations. In this embodiment, a portion of hand interface 123 is incorporated into sleeve 244, such as by thermally connecting (e.g., welding), chemically connecting (e.g., epoxying), or mechanically connecting (e.g., threading) hand interface 123 to sleeve 244, or by manufacturing sleeve 244, such as by casting or machining, to include hand interface 123.

As shown in the exploded view of FIG. 18, sleeve 244 may have a distal end 249 configured for threaded connection to a portion of a collar, a central bore 250 aligned with the longitudinal axis and having an inner diameter such that inner shaft 251 may be inserted and rotated in sleeve 244.

Proximal end of sleeve may have two or more slots 87 cut to form two or more proximally extending tangs 265, and which may also have a hand interface 123 to provide improved grip, less (hand) muscle strain, and increased mechanical advantage for a surgeon.

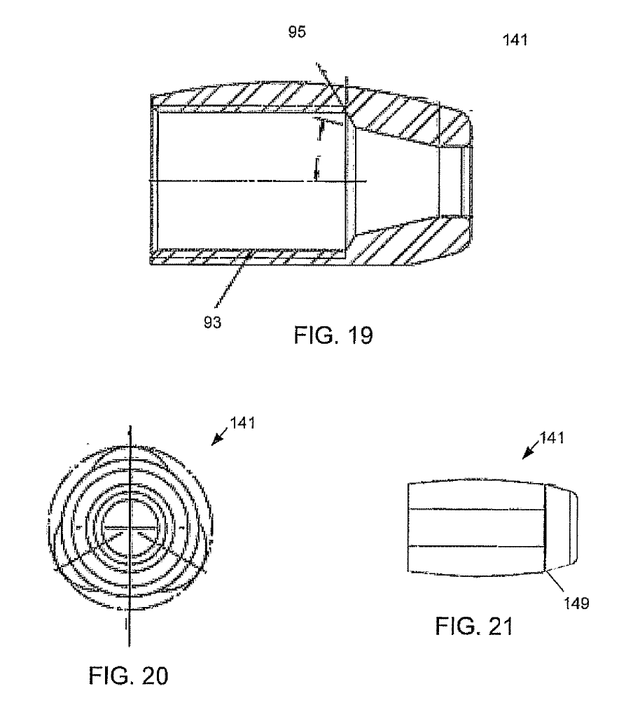

FIG. 19 depicts a cross-sectional view of a collet nut 141 for use in a collet-style locking mechanism. Collet nut threads 93 on collet nut 141 may engage collet threads on a sleeve. A tapered inner surface 95 on collet nut 141 may contact proximally extending tangs on sleeve to radially compress tangs to engage inner shaft. When an inner shaft is inserted into a sleeve and collet nut 141 is threaded far enough onto a collet thread, tangs such as tangs 265 in FIG. 18 may flex inward onto the inner shaft with sufficient force to frictionally connect the inner shaft to the sleeve at a proximal end of sleeve and inner shaft to prevent independent rotation of the inner shaft or outer sleeve.

Collet nut 141 may be manufactured from steel or steel alloys, titanium or titanium alloys, or other biocompatible materials. In a preferred embodiment, collet nut 141 may be manufactured from 455 SS (Stainless Steel), H900 or 17-4 phSS, H900. Collet threads 144 have selected thread length and thread count and may be a standard profile, for example ½-20 UNF-2A, for engagement with collet threads such as collet threads 129 in FIG. 18.

In FIG. 20, an end view of a collet nut 141 is shown in which the collet nut 141 may be symmetric about the longitudinal axis such that rotation of collet nut 141 on collet threads (such as collet threads 129 in FIG. 18) results in an unbiased compression force by a tapered inner surface on one or more tangs.

FIG. 21 depicts a side view of a collet nut 141. Tapered outer surface 149 may be useful for reducing (hand) muscle strain on the surgeon, providing an additional benefit over prior art devices.

During a minimally invasive surgical procedure, a plane may be created in tissue from a first vertebra to a second vertebra. An elongated member may be positioned in the plane during the surgical procedure. In some embodiments, a tissue plane may be formed using a targeting needle. The targeting needle may be positioned at the first vertebra. The distal end of the needle may be moved toward the second vertebra to form the plane while maintaining a position of the needle at a surface of the skin. The needle may be moved back and forth a number of times to clearly establish the plane. Care may need to be taken to avoid bending the targeting needle during establishment of the plane.

Minimally invasive procedures may involve locating a surgical site and a position for a single skin incision to access the surgical site. The incision may be located above and between (e.g., centrally between) vertebrae to be stabilized. An opening under the skin may be enlarged to exceed the size of the skin incision. Movement and/or stretching of the incision, bending of an elongated member, and angulation of collars of bone fastener assemblies may allow the length of the incision and/or the area of a tissue plane to be minimized. In some embodiments, minimally invasive insertion of a spinal stabilization system may not be visualized. In certain embodiments, insertion of a spinal stabilization system may be a top-loading, mini-opening, muscle-splitting, bone fastener fixation technique.

Insertion of a spinal stabilization system may include gradually increasing the diameter of an opening formed in a pedicle and/or vertebral body to accept a bone fastener assembly. For example, a targeting needle may have outer diameter of about D. A bone awl inserted after the targeting needle may have an outer diameter incrementally larger than the outer diameter of the targeting needle. As used herein, an incrementally larger diameter may be large enough to allow a snug but adjustable fit. For example, the bone awl may have outer diameter of about (D+x). A tap portion of a bone tap inserted after the bone awl may have a minor diameter of about (D+2x). A bone fastener may have a minor diameter of about (D+3x). In some embodiments, x may be between about 0.1 mm and about 1.0 mm. For example, x may be about 0.5 mm. Incremental sizing of the targeting needle, bone awl, tap, and bone fastener may promote a proper fit of the bone fastener in the vertebra to be stabilized.

In an embodiment of a spinal stabilization system insertion method, the patient may be placed in a prone position on a radiolucent table with clearance available for a C-arm of a fluoroscope. For example, a Jackson table with a radiolucent Wilson frame attachment may be used. The ability to obtain high quality images is very important. Bolsters, frames, and pads may be inspected for radiolucency prior to the operation. Placing the patient in a knee-chest position (e.g., using an Andrews table) should be avoided. Care should be taken to avoid placing the patient's spine in kyphosis during positioning of the patient.

The C-arm of the fluoroscope should be able to freely rotate between the anteroposterior, lateral, and oblique positions for optimal visualization of pedicle anatomy during the procedure. The arm should be rotated through a full range of motion prior to beginning the procedure to ensure that there is no obstruction or radio-opaque object in the way. The fluoroscope may be positioned so that Ferguson views and “bullseye” views are obtainable. Once the patient is positioned and the ability to obtain fluoroscopic images of the target levels for instrumentation has been confirmed, the patient may be prepared and draped sterilely.

For most of the lumbar region, the vertebral pedicle is an obliquely oriented cylindrical corridor. The angulation varies by approximately 5 degrees per level (e.g., Li: 5 degrees; L5: 25 degrees). A pre-operative fine-cut computed tomography image may be examined to determine any unique anatomy of the patient. Acquiring the pedicle in the most lateral and superior quadrant of the pedicle may be desirable to avoid the overriding facet during a minimally invasive procedure. A lateral entry point may allow for better bone fastener convergence as well as less interference with the superior adjacent level facet joint. A targeting needle may be passed in a medial and inferior trajectory, thus following the natural pathway of the pedicle. Frequent fluoroscopic inspection in both an anteroposterior and lateral plane may ensure proper passage of the needle as the needle is inserted into vertebral bone.

Various techniques may be used to plan the skin incisions and entry points. In one embodiment, the planning sequence for a single-level stabilization may include the following four steps. First, an anteroposterior image may be obtained with the spinous processes centered at the target vertebral bodies. Vertical lines passing through midpoints of pedicles that are to receive bone fasteners may be marked on the patient. The lines do not represent skin entry points. The lines are markers of pedicle entry points used to estimate angles at which targeting needles to be inserted to contact the pedicles. In some embodiments, sets of vertical lines may be drawn corresponding to the lateral edges of the pedicles instead of lines corresponding to the midpoints of the pedicles.

Second, horizontal lines may be marked approximately through the centers of the pedicles (mid-pedicle lines) on the patient. In some embodiments, the lines may be drawn on the superior side of the center axes (superior to the mid-pedicle).

Third, an oblique or “bullseye” view (i.e., down a longitudinal axis of a pedicle) may be obtained on each side of the patient for each pedicle that is to be stabilized. Vertical oblique view lines may be marked on the skin at the midpoints of each of the pedicles that are to receive a bone fastener. The oblique view lines may be drawn in a different color than the vertical lines drawn during the first step. In some embodiments, vertical lines may be drawn corresponding to the lateral edges of the pedicles instead of lines corresponding to the midpoints of the pedicles.

The oblique view lines may be about 2 cm to about 3 cm away from the lateral pedicle border lines marked in the first step. For larger patients, the oblique view line may be greater than about 3 cm away from the midline marked in the first step. For smaller patients, the oblique view line may be closer than about 2 cm away from the midline marked in the first step. The intersection of the oblique view lines with the horizontal lines drawn in the second step may represent skin entry points for a targeting needle as the targeting needle passes through soft tissue at an angle towards the bony pedicle entry point. A side fluoroscopic image, the horizontal lines, and the vertical lines may help the surgeon triangulate between the skin entry points and bony entry points.

Fourth, an incision may be made in the skin between mid-pedicle lines along the vertical oblique view lines. The skin incision may be from about 2 cm to about 4 cm long. In some embodiments, the incision may be from about 2.5 cm to about 3 cm long. Limiting the length of the incision may enhance patient satisfaction with the procedure. The incisions may be pre-anesthetized with, for example, 1% lidocaine with 1:200,000 epinephrine. To blunt the pain response, a long spinal needle may be used to dock on the bone entry point and inject the planned muscle path in a retrograde fashion as well. Once the incision has been made, tissue surrounding the incision may be pulled and/or stretched to allow access to a target location in a vertebra.

After sterile preparation and draping, the pedicle entry points may be fluoroscopically rechecked to ensure that the previously marked lines correspond to the intersection of the midline of the transverse process and the lateral joint and pars interarticularis. The intersection of the facet and the transverse process provides a starting point that may help avoid the canal and follow the natural inclination of lumbar pedicles. For the spinal stabilization system described, in which sleeves coupled to bone fastener assemblies are substantially unconstrained by insertion angles of the bone fasteners, patient anatomy may determine the most advantageous insertion angles of the bone fasteners.

A scalpel may be used to make a stab wound at the junction of an oblique view line and a mid-pedicle line. In an embodiment, the scalpel may be a #11 scalpel. A targeting needle may be passed through the incision in an oblique lateral to medial trajectory towards the bony entry point defined by a lateral pedicle border line. The C-arm of the fluoroscope may be placed in an anteroposterior position for this maneuver.

As a targeting needle encounters the bony anatomy, anteroposterior fluoroscopic images may be used to place the tip of the needle at the upper outer quadrant of the pedicle. In some embodiments, the needle may be walked medially along the transverse process to the pedicle entry point. In some embodiments, the needle tip may be docked by lightly tapping the tip into the bone with a mallet or other impact device to drive the tip into the bone. In some embodiments, the needle tip may be docked by applying downward pressure to the targeting needle to force the tip into the bone.

The fluoroscope may then be moved to a lateral position. The surgeon may correct the sagittal trajectory of the needle by moving the needle in an anterior or posterior direction to match the vector of the pedicle corridor. In some embodiments, a mallet or other impact device may be used to gently advance the targeting needle into the pedicle halfway to the pedicle-vertebral body junction. In other embodiments, force may be applied to the targeting needle to drive the targeting needle into the pedicle halfway to the pedicle-vertebral body junction. An anteroposterior image may then be obtained to confirm that the needle is approximately halfway across the pedicle in the anteroposterior view. If the tip is more than halfway across the pedicle in a lateral to medial projection, the trajectory may be too medial. Further advancement of the needle may risk passing the needle through the spinal canal. The needle may be repositioned. A new starting point or new trajectory may be obtained. If the anteroposterior image demonstrates that the needle is significantly lateral in the pedicle, then the needle may have passed along the lateral portion of the pedicle. A needle that has passed along the lateral portion of the pedicle may be withdrawn and repositioned.

Once a good trajectory has been obtained, the targeting needle may be advanced using a mallet. In some embodiments, the needle may be pushed in without a mallet. The targeting needle may be advanced to the junction of the pedicle and vertebral body under lateral fluoroscopic guidance. At this point, confirmation of position and trajectory should be repeated under anteroposterior fluoroscopy. The targeting needle may be further advanced to a desired depth within the vertebral body using a mallet or applied force.

A scale on the targeting needle may be used to approximate a length of a bone fastener to be used. A first depth of the targeting needle may be measured relative to the body surface when a pedicle is first encountered. A second depth of the targeting needle may be measured relative to the body surface after the targeting needle has been advanced to the desired depth in the vertebral body. An approximate length of the pedicle bone fastener to be used may be determined by taking a difference between the depth measurements.

After the targeting needle has been advanced into the bone, a portion of the targeting needle may be removed from the targeting needle. After removal of the member, a guide wire may be placed through a passage in the targeting needle into the vertebral body. Lateral fluoroscopic images may be obtained to indicate the position of the guide wire. In some embodiments, the guide wire may be pushed into the vertebral body. In certain embodiments, the guide wire may be advanced about 1 cm beyond an end of an outer housing to secure the guide wire in the vertebral body. In some embodiments, a small diameter tissue dilator may be placed over the guide wire and positioned on an upper surface of the targeting needle. The tissue dilator may provide stability to the guide wire. Added stability from the dilator may allow the guide wire to be successfully tapped into the vertebral body with a small mallet. Care should be taken to avoid kinking the guide wire. After the guide wire is secured in the vertebral body, the outer housing may be removed from the patient.

Once the guide wire has been passed through the targeting needle and the targeting needle has been removed, the guide wire may be used as a guide to position one or more successively sized dilators around a target location in a pedicle. A dilator may be a conduit with a regular shape (e.g., cylindrical) or an irregular shape (e.g., C-shaped). A dilator may form an opening through soft tissue to the pedicle. For patients with a thick fascia, it may be advantageous to make a nick in the fascia with a scalpel blade to facilitate passage of the dilators. The dilators may be passed sequentially over the guide wire. The dilators may be rotated during insertion to facilitate dilation of surrounding tissue. The dilators may be inserted until the leading edges contact the pedicle. A distal end of a dilator may be tapered to facilitate positioning of the dilator proximate the pedicle. An instrumentation set for a spinal stabilization system may include two, three, four, or more successively sized dilators.

After tissue dilation has been achieved, a bone fastener assembly driver having a sleeve rigidly connected to a bone fastener assembly and an inner shaft may be used to guide the bone fastener assembly toward a target location in a pedicle. A bone awl may be positioned over the guide wire in a dilator such that a tip of the bone awl is on or near a surface of a pedicle. The bone awl may be driven downwards into the pedicle to breach cortical bone of the pedicle. After the pedicle is breached, the bone awl may be removed from the dilator. In some embodiments, an initial passage may be formed in the pedicle and the vertebral body using a drill or a drill and tap combination.

The chosen bone fastener assembly may be attached to a sleeve having a movable member or collet-style locking mechanism. The movable member or collet-style locking mechanism may couple the inner shaft to the sleeve at a proximal end. A bone bone fastener or other bone fastener may then be connected to the collar (if not already attached) by top-loading the bone fastener through a central bore or alternatively through an opening in the side of the sleeve and seating the bone fastener in the collar, or the bone fastener may be bottom loaded into the collar.

When the bone fastener assembly is coupled to the sleeve, a drive portion of an inner shaft may be coupled to a tool portion of the bone fastener. In one embodiment, a collet nut may be positioned on the sleeve by axially aligning the collet nut with a proximal collet thread and rotating the collet nut until the collet nut threads engage at least a portion of the collet threads. An inner shaft may then be inserted into the sleeve such that the distal end of the inner shaft contacts the top surface of the bone fastener. The inner shaft may then be rotated inside the central bore of the sleeve to align engaging portions on the inner shaft with attachment features on the bone fastener. Once the distal end of the inner shaft has attached to the head of the bone fastener, the collet nut may be rotated to fully engage collet nut threads onto the collet threads, and may be further rotated until the axial movement of the collet nut results in a tapered inner surface on the collet nut contacting proximally extending the tangs on the sleeve to radially compress the tangs to engage the inner shaft. In this configuration, the action of threading the collet nut onto the collet thread may frictionally connect the inner shaft to the sleeve such that when the inner shaft is rotated, the sleeve, the collar, and the bone fastener also rotate as a single rigid unit to thread the bone fastener into bony tissue. A removable handle may be attached to the proximal end of inner shaft. The sleeve, collar, and bone fastener may be substantially co-axial when the fastener driver is positioned in the sleeve. In some embodiments, the removable handle may be attached to the inner shaft after the bone fastener, collar, sleeve, and inner shaft combination have been positioned down a guide wire through a dilator and against a pedicle.

In one embodiment, a movable member may be inserted in a passage or opening aligned transverse to the longitudinal axis of a sleeve. Using embodiments of the present disclosure the surgeon is able to implant a bone fastener into bony tissue while the bone fastener is connected to a collar. As an example, the distal end of a sleeve may be threaded onto a collar to engage sleeve with the collar. A bone bone fastener or other bone fastener can then be connected to bone fastener (if not already attached) by top-loading a bone fastener through the bore or alternatively through openings in the side of sleeve and seating bone fastener in collar, or the bone fastener may be bottom loaded into the collar. A movable member may pass through an opening in sleeve. The movable member may be slidably positioned in passage of sleeve so that a first section of movable member is axially aligned with bore of sleeve. An inner shaft may be inserted into the sleeve such that distal end of the shaft contacts a portion of the bone fastener. The inner shaft may be rotated inside the bore of the sleeve to align the inner shaft with the head of the bone fastener. Once the distal end of the inner shaft has engaged the head of the bone fastener, the movable member may be moved between two positions in one or more openings in sleeve such that the second section of the movable member is axially aligned with the central bore of sleeve and the inner shaft is captured by the movable member. In this configuration, the movable member has fixedly connected the inner shaft to the sleeve such that when the inner shaft is rotated, the sleeve, the collar, and the bone fastener also rotate as a single rigid unit to thread the bone fastener into bony tissue. Advantageously, embodiments of the present disclosure enable surgeons to selectively change the configuration of the device by sliding or otherwise moving a locking device from a first position to a second position, without the use of tools. Advantageously, embodiments of the present disclosure in which an shaft and a sleeve rotate as a single assembled unit enable surgeons to surgically penetrate the patient at a selected site using MIS procedures to implant a collar and a bone fastener at the same time without fear of disconnection or loosening of either the bone fastener or the collar.

In one embodiment of a procedure for inserting a bone fastener assembly into a patient, an inner shaft coupled to a bone fastener may be coupled to a sleeve which is coupled to a collar of a bone fastener assembly. The bone fastener assembly driver may be inserted along a guide wire into a dilator. In some embodiments, tissue surrounding the incision may be pulled and/or stretched to allow a desired angular orientation of the bone fastener assembly relative to the pedicle. After insertion of the bone fastener assembly driver in the dilator, the components of the bone fastener assembly driver (i.e., the inner shaft, sleeve, collar, bone fastener, and movable member or collet-style mechanism) may be rotated as a single unit to thread the bone fastener into the pedicle and the vertebral body. The bone fastener (while coupled to the collar) may be advanced into the pedicle under fluoroscopic guidance to inhibit breaching of the pedicle walls. When the tip of the bone fastener advances beyond the posterior margin of the vertebral body, the guide wire may be removed to inhibit inadvertent bending of the guide wire or unwanted advancement of the guide wire.

The bone fastener assembly may be advanced to bring the collar down snug to the facet joint. The bone fastener may then be backed off about a quarter of a turn. Backing the fastener off about a quarter of a turn may allow for full motion of the collar relative to the bone fastener. Once the bone fastener is implanted in the bony tissue, the surgeon may remove the inner shaft and sleeve from the patient. In one embodiment, once the bone fastener has been implanted in the bony tissue, the surgeon may rotate the collet nut in a reverse direction such that collet nut threads partially disengage from the collet threads to release the radial pressure on the tangs, and the tangs are free to expand into a configuration whereby the inner shaft is rotatably connected and therefore free to rotate independent of the sleeve. The sleeve may then be rotated in a reverse direction to disengage threads on the distal end from threads on the collar, and the inner shaft and the sleeve may be removed from the patient, leaving the bone fastener and collar securely implanted in bony tissue at the selected site. In some embodiments, the inner shaft may be removed from the sleeve by withdrawing inner shaft from sleeve, and in other embodiments, the inner shaft may be removed from the sleeve by allowing the inner shaft to pass through the sleeve because the sleeve is no longer connected to a collar. The collet nut may be removed from the collet threads by continuing to rotate collet nut in a reverse direction to disengage the collet nut threads from the collet threads.

In another embodiment, once the bone fastener and collar have been implanted in the bony tissue, the surgeon may position the movable member into the first position such that the first section is axially aligned with shaft and sleeve, whereby the inner shaft may be free to rotate independent of the sleeve. The sleeve may then be rotated in a reverse direction to disengage threads on the distal end from threads on the collar, and the inner shaft and sleeve may be removed from the patient, leaving the bone fastener and the collar securely implanted in bony tissue at the selected site. In some embodiments, the inner shaft may be removed from the sleeve by withdrawing the inner shaft from the outer sleeve, and in other embodiments, the inner shaft may be removed from the sleeve by allowing the inner shaft to pass through the sleeve because the sleeve may be no longer connected to a collar. A movable member may be removed by withdrawing it from an opening or, in embodiments having two openings positioned opposite each other on a sleeve, may be removed by either withdrawing it through a first opening or by passing it through a second opening located opposite the first opening.

With bone fastener assemblies secured in the vertebral bodies, sleeves coupled to the bone fastener assemblies may be oriented to facilitate insertion of an elongated member in the sleeves. In some embodiments, sleeves may serve as tissue retractors during a spinal stabilization procedure. Angular motion of a collar may be limited by a range of motion allowed between the collar and the bone fastener that the collar is bone anchored to. Angular motion of a collar may be limited by patient anatomy. Angular motion or orientation of one collar (i.e., sleeve), however, may not depend upon a position of another collar (i.e., sleeve). In some embodiments, channel openings in the sleeves may face each other. In other embodiments, channel openings in the sleeves may be angled relative to each other in various arrangements. A distance between the sleeves may be estimated using an estimating tool. The distance between the sleeves may be used to select a length of an elongated member needed to couple the collars.

A spinal stabilization system may be used to stabilize two or more vertebral levels (i.e., at least three adjacent vertebrae). In an embodiment, an incision may be made in the skin between the outermost vertebrae to be stabilized. A first bone fastener assembly may be coupled to a first sleeve and a first inner shaft. The first bone fastener assembly may be threaded into a first pedicle at a target location and the inner shaft removed such that the first sleeve extends above the body surface. The first sleeve may rotate about the head of the first bone fastener. A tissue plane may be created between a channel opening in the first sleeve and a target location at a second pedicle. In an embodiment, the second pedicle may be adjacent to the first pedicle. A second bone fastener assembly may be coupled to a second sleeve and inner shaft and threaded into the second pedicle through the incision. Another tissue plane may be created between the first sleeve or the second sleeve and a target location in a third pedicle. The third pedicle may be adjacent to the first pedicle and/or the second pedicle. A third bone fastener assembly may be coupled to a third sleeve and inner shaft and threaded into the third pedicle through the incision.

In an embodiment of a method for a two-level spinal stabilization procedure, an incision may be made above a target location in a middle pedicle. A first bone fastener assembly may be anchored to the middle pedicle. After the first bone fastener assembly is secured, second and third bone fastener assemblies may be coupled to outer pedicles as desired by pulling and/or stretching tissue surrounding the incision to allow access to the outer pedicles.

After an elongated member has been positioned and seated in collars as desired, closure members may be used to secure the elongated member to the collars. One or more counter torque wrenches may be used during shearing of the tool portions of the closure members.

In certain embodiments, an external frame may be used to impose a desired constraint on one or more sleeves. For example, an external frame may hold one or more sleeves in a particular location and/or orientation such that a desired relative positioning of vertebrae may be achieved. An external frame may be used to impose a distance and/or angle between sleeves to achieve distraction or compression of vertebrae. Reduction of vertebrae may be achieved when an external frame is used to adjust a relative height of the sleeves.

In some embodiments, a spinal stabilization system may be inserted using an invasive procedure. Since insertion of a spinal stabilization system in an invasive procedure may be visualized, cannulated components (e.g., bone fasteners) and/or instruments (e.g., sleeves) may not be needed for the invasive (i.e., open) procedure. Thus, a bone fastener used in an invasive procedure may differ from a bone fastener used in a minimally invasive procedure.

Embodiments of the present disclosure enable surgeons to selectively configure an inner shaft and sleeve to either rotate independently or rotate as a single unit. Allowing the inner shaft and sleeve to rotate independently allows the surgeon to connect to, manipulate, and disconnect from either the bone fastener or the collar. Advantageously, embodiments of the present disclosure may be usefully applied to threading bone screws with attached collars in minimally invasive surgeries (MIS) due to the ease of manipulation of the device attachment. The inner shaft may be inserted and locked to the outer shaft, and unlocked and removed from the outer shaft using hand operations only (i.e., no other tools.)

A further benefit to having an inner shaft that may be rigidly connected is that once the inner shaft has been locked to the outer shaft, the bone fastener and collar are retained in a rigid configuration that prevents partial disconnection that could lead to off-axis rotations (i.e. precession) by the bone fastener resulting in bony tissue damage and injury to the patient, or damage to the collar which could prevent proper attachment to the bone fastener or proper connection to a rod. Furthermore, partial disconnection may lead to complete disconnection from the collar or bone fastener, which could be harmful to the patient and extends the time in surgery to locate and retrieve a lost screw, bone fastener, or collar. In minimally invasive spine surgeries, the bone fastener assembly driver must not accidentally disconnect from the bone fastener, and equally important, the bone fastener assembly driver must not partially disconnect or loosen from the bone fastener. A bone fastener that has loosened from the device could miss the target and hit the spinal cord, a disc, nerve, or artery, or damage the hole from the effects of precession such that implantation is not possible.

In some embodiments, tools used in an invasive procedure may be similar to tools used in a minimally invasive procedure. In certain embodiments, methods of installing a spinal stabilization system in an invasive procedure may be similar to methods of installing a spinal stabilization system in a minimally invasive procedure.

In the foregoing specification, the disclosure has been described with reference to specific embodiments. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

What is claimed is:

1. A bone fastener assembly driver for implanting in bony tissue a bone fastener connected to a collar, comprising:
a sleeve defining a central bore aligned with the longitudinal axis of the sleeve and at least one opening normal to the central bore, the sleeve comprising a distal end configured for threaded connection to a collar;
an inner shaft configured for insertion into the central bore, comprising a distal end configured for connection to the bone fastener; and
a proximal end configured for selective engagement with a tool, a movable member configured for slidable positioning in two positions in the at least one opening normal to the central bore, having
an elongated hole comprising
a first section configured to allow rotation of the inner shaft, and
a second section configured to inhibit rotation of the inner shaft;
wherein slidably positioning the movable member in the sleeve such that the inner shaft passes through the first section of the movable member maintains the inner shaft in rotatable connection with the sleeve, and slidably positioning the movable member in the sleeve such that the inner shaft passes through the second section of the movable member engages the inner shaft in fixed connection with the sleeve.

2. The bone fastener assembly driver of claim 1, wherein the distal end of the inner shaft is configured for connection to a key-style bone fastener feature.

3. The bone fastener assembly driver of claim 1, wherein the distal end of the inner shaft is configured for connection to an internal hex bone fastener feature.

4. The bone fastener assembly driver of claim 1, wherein when the movable member is in the first position the inner shaft is axially aligned with the first section and the sleeve and when the movable member is in the second position the inner shaft is axially aligned with the sleeve and second section.

5. A method comprising:
rotating a sleeve about its longitudinal axis such that a threaded portion of the distal end engages a threaded portion of a collar, wherein the sleeve defines a central bore aligned with the longitudinal axis of the sleeve and at least one opening normal to the central bore;
slidably positioning a movable member in a first position in the at least one opening normal to the central bore, wherein the movable member defines
an elongated hole having
a first section of first diameter, and
a second section of second diameter communicably coupled to the first section; and
inserting an inner shaft through the central bore of the sleeve such that the inner shaft passes through the first section,
wherein the inner shaft comprises:
a distal end configured for engagement with a portion of the bone fastener; and
a proximal end configured for selective engagement with a tool;
moving the movable member from the first position to the second position such that the inner shaft passes through the second section to inhibit relative rotation of the inner shaft relative to the sleeve.

6. The method of claim 5, wherein the distal end of the inner shaft is configured for connection to a key-style bone fastener feature.

7. The method of claim 5, wherein the distal end of the inner shaft is configured for connection to an internal hex bone fastener feature.

8. The method of claim 5, further comprising:
attaching the distal end of the inner shaft to a portion of the bone fastener.

9. The method of claim 5, further comprising:
entering a body with the bone fastener, collar, and a portion of the inner shaft and portion of the sleeve.

10. The method of claim 5, further comprising:
rotating the sleeve, wherein the inner shaft, collar, and bone fastener also rotate such that the bone fastener threads into bony tissue at a selected site.

11. The method of claim 5, further comprising the steps of:
slidably positioning the movable member from the second position to the first position such that the inner shaft is rotatable relative to the sleeve;
rotating the sleeve in a reverse direction to disengage the distal end threads from the collar threads; and
extracting the sleeve and inner shaft from the body, wherein the bone fastener and collar are connected to the bony tissue.

12. The bone fastener assembly of claim 5, wherein the distal end of the inner shaft is configured for connection to a key-style bone fastener feature.

13. The bone fastener assembly of claim 5, wherein the distal end of the inner shaft is configured for connection to an internal hex bone fastener feature.

14. A bone fastener assembly driver for implanting in bony tissue a bone fastener connected to a collar, comprising:
an inner shaft comprising:
a distal end configured for engagement with a portion of the bone fastener; and
a proximal end configured for selective engagement with a tool;
a sleeve defining a central bore, the sleeve comprising:
a distal end configured for connection to a collar; and
a proximal end configured with a plurality of slots radially disposed about the longitudinal axis to form a plurality of proximally extending tangs; and
a collet thread; and
a collet nut having a tapered inner surface and a thread for engagement with the collet thread,
wherein the action of threading the collet nut onto the collet thread radially compresses the plurality of tangs inward to frictionally engage the inner shaft.

15. The bone fastener assembly of claim 14, wherein the bone fastener comprises an internal hex feature.

16. The bone fastener assembly of claim 14, wherein the bone fastener comprises a key-style profile feature.

17. A method comprising:
rotating a sleeve about its longitudinal axis such that a threaded portion of the distal end engages a threaded portion of the collar, wherein the sleeve further comprises:
a central bore aligned with the longitudinal axis of the sleeve;
a plurality of slots radially disposed about the longitudinal axis to form a plurality of proximally extending tangs having selected spring constant; and
a collet thread;
inserting an inner shaft through a central bore of the sleeve;
wherein the inner shaft comprises:
a distal end configured for engagement with a portion of the bone fastener; and
a proximal end configured for selective engagement with a tool;
connecting the distal tip of the inner shaft to an attachment profile on the bone fastener;
rotating a collet nut comprising a thread to engage the collet thread, wherein the collet nut comprises a tapered inner surface such that continued threading of the collet nut on the collet thread at least partially contacts the collet nut tapered inner surface to the plurality of tangs to radially compress the plurality of tangs such that the plurality of tangs frictionally engages the inner shaft.

18. A method for implanting a bone fastener in bony tissue comprising:
inserting a portion of a bone fastener having selected profile into a collar;
threading the collar onto the distal end of a sleeve, wherein the distal end is configured for attachment to a collar, such that the collar seats on the sleeve,
wherein the sleeve further comprises:
a proximal end configured for connection to a surgical tool;
a plurality of slots radially disposed about the longitudinal axis to form a plurality of proximally extending tangs having selected spring constant;
a central bore aligned with the longitudinal axis of the sleeve and configured for selective engagement of the inner shaft; and
a collet thread;
inserting an inner shaft through a central bore of the sleeve aligned with the longitudinal axis of the sleeve;
wherein the inner shaft comprises:
a distal end configured for connection to a portion of the bone fastener; and
a proximal end configured for selective engagement with a tool;
connecting the distal tip of the inner shaft to an attachment profile on the bone fastener; and
rotating a collet nut comprising a thread to engage the collet thread, wherein the collet nut comprises a tapered inner surface such that continued threading of the collet nut on the collet thread at least partially contacts the collet nut tapered inner surface to the plurality of tangs to radially compress the plurality of tangs such that the inner shaft is frictionally connected to the sleeve.

19. The method of claim 18, further comprising:
entering a body with the bone fastener, collar, and a portion of the inner shaft and portion of the sleeve.

20. The method of claim 18, further comprising:
rotating the sleeve, wherein the inner shaft, collar, and bone fastener also rotate such that the bone fastener threads into bony tissue at a selected site.

21. The method of claim 18, further comprising the steps of:
rotating the collet nut in a reverse direction such that at least part of the collet nut thread disengages at least part of the collet thread to release the forces exerted by the collet nut tapered inner surface on the plurality of tangs;
rotating the sleeve in the reverse direction such that the thread on the distal end of the sleeve disengages the threads on the collar; and
withdrawing the portion of inner shaft and portion of the outer shaft from the body.
